(19) United States
(12) Patent Application Publication
(10) Pub. No.: US 2025/0195486 A1
(43) Pub. Date: Jun. 19, 2025

(54) EDOXABAN DOSING

(71) Applicant: Closed Loop Medicine Ltd., Cambridge (GB)

(72) Inventors: Paul Goldsmith, London (GB); Bruce Campbell, Dover (GB); Michael Catt, Wellingborough (GB); Tom Godec, London (GB); Joe Coghlin, London (GB)

(73) Assignee: Closed Loop Medicine Ltd., Cambridge (GB)

(21) Appl. No.: 18/541,362

(22) Filed: Dec. 15, 2023

(51) Int. Cl. A61K 31/444; G16H 10/60; G16H 20/10

(52) U.S. Cl. CPC A61K 31/444; G16H 10/60; G16H 20/10

(57) ABSTRACT

A computer implemented method for determining a dosage of edoxaban for administering to a patient, the method comprising: receiving patient data relating to a patient, wherein the patient data includes a kidney function metric; processing the patient data with a dosage calculator to determine the dosage of edoxaban for administering to the patient, wherein the dosage calculator is derived from a plasma level prediction model; and indicating the dosage.

### FIELD

The present disclosure relates to a method for determining a dosage of edoxaban, a method for generating a dosage calculator and a dosage calculator.

### BACKGROUND

The body's clotting system has evolved to a particular equilibrium. This represents a trade-off. Clotting is a protective factor to repair internal blood vessel breakdown or external wounds. However, too much tendency to clot and blood vessels become blocked when this is not desired. This clot can then break off and embolize to distant blood vessels with catastrophic consequences. There is a constant turnover of the multiple molecular components involved in clotting.

Clots can form in any part of the vasculature, in particular the veins of the leg, arteries of the thorax and neck and within the heart. Clots that either embolize from the heart or neck arteries or form directly within the cerebral vasculature can cause a stroke.

Clots are more likely to form in the heart if the heart chambers are enlarged or do not contract normally. Atrial fibrillation (AF), characterized by disorganized atrial electrical activation and contraction in the heart muscle, accounts for ˜30% of all hospitalisations for heart rhythm irregularities, occurring in almost 10% of people over the age of 80 with prevalence increasing as the population gets older. The clinical consequences of uncontrolled AF results in a 5-fold increase in stroke and blood clots requiring hospitalisation with consequential increasing health costs which in the USA alone is currently estimated to be $8 billion per year.

Management of AF includes methods to restore normal sinus rhythm, control heart rate, and where possible prevent recurrence. When these methods do not succeed, anticoagulants are used to inhibit the formation of the clots. In the past, warfarin has been successfully used, significantly reducing stroke by about 60%. However, warfarin can lead to severe risks of bleeding due to a narrow therapeutic range, differences in metabolism between individuals, and multiple interactions with a number of co-administered drugs and foodstuffs. Warfarin is difficult to prescribe at the correct dose and demands frequent measurements of blood clotting times (international normalised ratio (INR) levels) to allow for regular dose adjustment.

Another disease in which anticoagulants are used is in the treatment of thromboembolism where clots are formed in the veins (VTE, or venous thromboembolism). If untreated, VTE can lead to disability (pain, scaling ulcers, oedema in the legs), deep vein thrombosis (DVT), and if clots break off can cause pulmonary embolism and death. As many as 600,000 VTEs occur each year in the USA.

New drugs have been developed which allow simplified dose management and have been shown to reduce the chance of developing a major bleeding event when compared to warfarin. These direct acting oral anticoagulants (DOACs) act through different more targeted mechanisms. Whereas warfarin and other similar anticoagulants are indirect inhibitors of vitamin K through both intrinsic and extrinsic pathways, DOACs work directly on the common pathways lower in the clotting cascade. For example, one DOAC, edoxaban, acts directly by inhibiting the thrombin molecule both in free and bound forms. Thrombin is central to the formation of blood clots. These newer modes of action lead to fewer monitoring requirements, less frequent follow-ups, more immediate drug onset and offset effects (particularly important in relating plasma drug levels to activity), and fewer drug and food interactions.

Edoxaban is a DOAC licensed for stroke prevention in non-valvular atrial fibrillation, as well as the treatment and prevention of VTE; it has been studied in many thousands of patients. The ENGAGE AF-TIMI 48 trial, a phase III randomised, double-blind trial comparing two once-daily regimens of edoxaban with warfarin in 21,105 patients with moderate-to-high-risk atrial fibrillation, showed that edoxaban was superior to warfarin (dose-adjusted to achieve an international normalisation ratio INR of 2.0 to 3.0) in reducing the outcome of stroke or systemic embolic events rand major bleeding, with the advantage of a small reduction in the risk of intracranial bleeds. Edoxaban is also used to reduce the tendency to form clots and thus the incidence of stroke in patients from other systemic embolisms.

There are disadvantages to the DOACs such as edoxaban, including lack of efficacy and safety data in patients with severe chronic kidney or hepatic disease or those with significant valvular disease, lack of easily available monitoring of blood levels and compliance, and higher patient cost in some healthcare areas. Additionally, while reversal agents are now available for some DOACs, they are expensive and do not cover all forms of bleeding.

There are three doses of edoxaban available in the USA and Europe (15, 30, and 60 mg) to be taken once daily. Prescriptions may mix tablet doses to achieve the recommended posology for individuals who may not meet the normal criteria.

The limitations in dosage quanta and the prescribing guidelines can result in very limited dosing flexibility for a healthcare professional (HCP). As a result, patients can be prescribed an inappropriate starting dose and some patients may be excluded from treatment with edoxaban, for example those with kidney impairment. Real-world evidence studies by the US Food and Drug Administration (FDA) have provided evidence that currently DOACs may be both underdosed, leading to an excess of thrombotic events, and overdosed, leading to an excess of haemorrhage. Overdosing is particularly common in patients with renal impairment.

The issue of incorrect starting dosage (or even a correct starting dosage) can be further exacerbated by insufficient monitoring. Unlike warfarin, routine monitoring of blood coagulation in patients taking DOACs is not currently recommended, except in certain patients, particularly those with cryptic thromboses, renal failure, the elderly, or those taking certain coadministered drugs. In the latter groups, monitoring is needed but not often undertaken. Recommendations are that such patients should be reviewed at least once per year. This recommendation is often poorly adhered to and often no review is performed at all. Reasons include delegation to general practitioners and general physicians who may be too busy and/or do not have sufficient information and understanding. Furthermore, even when haematology experts give clear instructions to primary care, the instructions are often not followed properly.

A further challenge resulting from insufficient monitoring is managing risk around the times of invasive procedures for an operation, such as a hip/knee replacement or a lumbar puncture. Management of patients in the perioperative period involves a careful assessment of the relative risk of bleeding or the possibility of a thromboembolic event. Current guidelines are a one-size-fits-all with the result that some patients have their anticoagulation stopped too soon and are thus rendered at high risk of clots, whereas others may have their anticoagulation stopped too late and have higher bleeding risks. As well as risks to the patient, there is also secondary harm from bed blocking from excessive stays in hospital while waiting for anticoagulation to wear off. Furthermore, there is also delay in any investigations. For example, an unplanned lumbar puncture, which may be required at short notice to diagnose a neurological condition, may be delayed for an unnecessarily long period because of concerns around ongoing anticoagulation.

A further area of particular need relates to thrombosis risk in cancer. Management is particularly difficult in cancer because, as well as an increased thrombosis risk, there is also an increased bleeding risk. The problems with cancer are increasing with the shift to home-based chemotherapy and thus more chemotherapy lines being inserted, further increasing the thrombosis risk. The requirement for particularly precise control and knowledge of actual risks of haemorrhage would be extremely useful.

The present disclosure provides a method for determining a dosage of edoxaban, a method for generating a dosage calculator and a dosage calculator that may address one or more of the above issues.

### SUMMARY

According to a first aspect of the present disclosure there is provided a computer implemented method for determining a dosage of edoxaban for administering to a patient, the method comprising:
receiving patient data relating to a patient, wherein the patient data includes a kidney function metric;
processing the patient data with a dosage calculator to determine the dosage of edoxaban for administering to the patient, wherein the dosage calculator is derived from a plasma level prediction model; and
indicating the dosage.

The patient data may further comprise one or more of: a patient age; a patient ethnicity; a patient sex (or patient gender); a patient weight; a patient genotype; a patient cardiac metric; and a patient medication list.

The patient cardiac metric may comprise an indication that the patient has non-valvular atrial fibrillation.

The patient genomic type may comprise a patient genotype for Pgp transporter genes such as ABCG or metabolic enzymes such as CYP 3A4/5.

The patient medication list may comprise an indication of whether the patient is consuming one or medications comprising: a proton-pump inhibitor; a calcium channel blocker; a P-gp inhibitor; an antifungal; an antiarrhythmic drug; an antibiotic; or a drug that increases a bleeding risk including a drug with an anticoagulant effect.

The one or more medications may comprise one or more of:
a proton pump inhibitors such as esomeprazole;
an antifungal such as ketoconazole;
an antiarrhythmic such as amiodarone or dronedarone;
an antibiotic such as rifampicin;
a drug that increases a bleeding risk such as a nonsteroidal anti-inflammatory drug, aspirin or clopidogrel.

The patient data may further comprise one or more of: reported side-effects; alcohol intake; smoking history, a patient clotting metric; a treatment purpose; patient genetic determinants; patient co-conditions; a patient activity level; a patient dosage compliance; a patient liver function; a patient thrombosis history; a patient haemorrhage history; a patient cancer history; a family thrombosis history; familial stroke history, familial bleeding history; a patient cardiovascular history; a patient metabolic history; a patient blood pressure history; a patient platelet count; a patient heart rate; and a patient haematocrit.

The treatment purpose may comprise: prevention of thrombosis, embolism and/or stroke, optionally including patients with non-valvular atrial fibrillation and one or more risk factors including: a previous stroke or transient ischaemic attack, heart failure, diabetes or hypertension; active thrombosis treatment and/or active pulmonary embolism treatment; prevention of venous thromboembolism in people who have undergone surgery, optionally including hip or knee replacement therapy.

The method may further comprise:
receiving updated patient data; and
processing the updated patient data with the dosage calculator to determine an updated dosage; and
indicating the updated dosage.

The updated patient data may include a patient clotting metric and/or a drug concentration, from a blood test result.

The method may further comprise calibrating the dosage calculator by adjusting the dosage calculator and/or the plasma level prediction model using the patient clotting metric and/or drug concentration.

The plasma level prediction model may comprise a time-based differential equation model for modelling a time dependence of a plasma concentration of edoxaban as a function of the patient data.

Processing the patient data with a dosage calculator to determine the dosage of edoxaban for administering to the patient may comprise:
receiving a target plasma level metric; and
calculating the dosage for administering to the patient by processing the patient data and the target plasma level metric with the dosage calculator.

Processing the patient data with a dosage calculator to determine the dosage of edoxaban for administering to the patient may comprise:
setting an initial value of a dose estimate;
processing the dose estimate with the dosage calculator to estimate a plasma level metric;
comparing the plasma level metric to a target plasma level metric; and
determining the dosage for administering to the patient by refining the dose estimate based on the comparison.

Refining the dose estimate may comprise:
iteratively adjusting the dose estimate and recalculating the plasma level metric until a difference between the estimated plasma level metric and the target plasma level metric is less than a difference threshold; or
   setting one or more further values of the dose estimate and processing the one or more further values with the dosage calculator to estimate one or more further plasma level metrics; and
   determining the dosage for administering to the patient by interpolating the dosage value corresponding to the target plasma level metric based on the relationship between the initial value of the dose estimate, the one or more further values of the dose estimate, the plasma level metric and the one or more further plasma level metrics.

The target plasma level metric may comprise one or more of:
a target trough plasma level comprising an ideal therapeutic level;
   a target maximum plasma level being less than a maximum level threshold;
a target average plasma level over a dosing interval at steady-state comprising an ideal therapeutic level;
   a target area under the curve of a plasma level time profile comprising an ideal therapeutic level;
   a target ratio of a maximum plasma level to a trough plasma level comprising an ideal therapeutic ratio and level; or
   a target ratio of the maximum plasma level to the area under the curve of the plasma level time profile comprising an ideal therapeutic ratio and level.

The patient data may comprise one or more target dependent patient parameters comprising: reported side effects; a patient thrombosis history; a patient haemorrhage history; a patient cancer history; a patient stroke history; a patient liver function metric; a patient heart function metric; a patient brain state; a patient smoking history; a patient alcohol history; a patient blood pressure; a patient activity level; a patient dosage compliance; a patient mobility state; a patient menstruation state; a patient inflammation state; a patient infection state; a patient comedication; a patient co-condition; a blood clotting metric; a patient genetic profile; familial stroke history, familial bleeding history; familial hypertension history; a patient cardiovascular history; a patient metabolic history; a patient blood pressure history; a patient blood pressure; a patient heart rate; a patient platelet count; a patient haematocrit; and a patient hydration state, wherein the method comprises:
determining the target plasma level metric as a personalised target plasma level metric based on the one or more target dependent patient parameters.

The method may comprise:
determining a bleeding risk score for the patient based on one or more of the target dependent patient parameters; and
determining a thrombosis risk score for the patient based on one or more of the target dependent patient parameters; and
determining the personalised target plasma level metric based on the bleeding risk score and/or the thrombosis risk score.

The method may comprise:
determining a bleeding risk score for the patient based on one or more of:
   a time since one or more bleeding risk events, wherein the bleeding risk events comprise any of: an injury; a surgical procedure; needle penetration; or a patient fall, as indicated by the patient haemorrhage history;
   a severity of the clotting risk event;
   an age of the patient;
   the patient cancer history;
the patient blood pressure;
   the patient liver function metric;
   a time since a haemorrhagic stroke as indicated by the patient stroke history;
   an extent of amyloid in the patient's brain as indicated by the patient brain state;
the blood clotting metric;
   an indication of aspirin, clopidogrel, NSAIDS, steroids, P-gp inhibitors, or antidepressants as indicated by the patient comedication;
   alcohol consumption as indicated by the patient alcohol history; and/or
determining a thrombosis risk score for the patient based on one or more of:
   a time since one or more clotting risk events, wherein the clotting risk events comprise any of:
a thrombosis; an injury; a surgical procedure; needle penetration; or a patient fall as indicated by the patient thrombosis history;
an episode of atrial fibrillation as indicated by the patient heart function metric;
an episode of chemotherapy as indicated by the patient cancer history;
   a severity of the clotting risk event;
   the patient cancer history;
the patient blood pressure;
the patient mobility;
   the patient blood pressure;
   the blood clotting metric;
   the patient genetic profile;
   the time since an ischaemic stroke as indicated by the patient stroke history;
   the patient menstruation state;
   an indication of oral contraceptive as indicated by the patient co-medication; and
   the patient hydration state,
wherein determining the personalised target plasma level metric is determined based on the bleeding risk score and/or the thrombosis risk score.

The personalised target plasma level metric may comprise:
a trough plasma level or an average plasma level, comprising a personalised adjustment to an ideal therapeutic level based on the one or more target dependent patient parameters; and/or
a ratio of a maximum plasma level to an area under a plasma level time profile being less than a bleeding risk threshold.

The ideal therapeutic level may comprise:
a trough plasma level range of 10-40 ng/ml;
a trough plasma level range of 20-30 ng/ml; or
a trough plasma level of 25 ng/ml]

The method may comprise:
processing the patient data with the dosage calculator to determine a plasma level metric; and
indicating one or more of:
   the plasma level metric;
   a patient thrombosis risk based on the plasma level metric; or
   a patient haemorrhage risk based on the plasma level metric.

The plasma level metric may comprise a plasma level time profile.

Indicating one or more of: the plasma level metric; the patient thrombosis risk; or the patient haemorrhage risk, may comprise indicating to the patient or a healthcare professional. Indicating may be via a user interface.

The patient data may comprise one or more dosage times at which the patient received a dose of edoxaban. The plasma level metric may comprise a time-dependent plasma level metric based on the one or more dosage times.

The plasma level metric may comprise one or more of:
a plasma level time profile;
a maximum plasma level;
a trough plasma level;
an average plasma level over a dosing interval at steady state;
an area under the curve of the plasma level time profile;
a ratio of the maximum plasma level to the trough plasma level; or
a ratio of the maximum plasma level to the area under the curve of the plasma level time profile.

The dosage calculator may comprise a machine learning algorithm trained using the plasma level prediction model.

The dosage calculator may comprise a machine learning algorithm trained using simulated population data obtained from the plasma level prediction model.

The dosage calculator may comprise a machine learning algorithm trained using:
simulated population data obtained from the plasma level prediction model; and real population data.

The machine learning algorithm may be locked to prevent further adjustment to the machine learning algorithm.

The machine learning algorithm may comprise an adjustable machine learning algorithm. The method may further comprise:
receiving updated patient data including a patient clotting metric from a blood test result; and
adjusting the machine learning model based on the patient clotting metric.

The dosage calculator may comprise the plasma level prediction model.

The dosage calculator may comprise one or more look-up tables defined according to simulated population data obtained from the plasma level prediction model.

Processing the patient data with the dosage calculator to determine the dosage of edoxaban for administering to the patient may comprise:
processing the patient data with the dosage calculator to determine an ideal dosage regime; and
selecting the dosage for administering to the patient from a selection of available dosage regimes based on the ideal dosage regime.

The selection of available dosage regimes may comprise dosage amounts comprising: 15, 30, 60 mg of edoxaban.

The selection of available dosage regimes may comprise dosage amounts comprising:
any multiple of 0.5 mg;
any multiple of 1 mg;
any multiple of 2.5 mg;
any multiple of 5 mg;
any multiple of 10 mg; or
any multiple of 25 mg.

The selection of available dosage regimes may comprise selection of a microgranular or liquid formulation for titrating an ideal dosage amount of the ideal dosage regimen.

Processing the patient data with the dosage calculator to determine the dosage of edoxaban for administering to the patient may comprise processing the patient data with the dosage calculator to determine one or more of: a dosage amount; a dosage time; a dosage frequency; and/or a dosage type.

The dosage type may comprise an edoxaban slow-release formulation with a specific release time.

The specific release time may be at least 6 hours.

Indicating the dosage may comprise indicating the dosage to a health care professional and/or to the patient.

Indicating the dosage may comprise indicating the dosage to a healthcare professional and/or the patient via a user interface. The user interface may comprise a digital app. the method may comprise performing one or more of the steps within the digital app.

Any method disclosed herein for use in stroke prevention, thrombosis treatment, blood clot prevention or dosage management for an invasive procedure on the patient.

The patient may be a cancer patient.

According to a second aspect of the present disclosure there is provided a computer readable medium comprising instructions which, when executed by one or more processors, cause the one or more processors to carry out any method disclosed herein.

According to a third aspect of the present disclosure, there is provided a method of generating a dosage calculator for determining a dosage of edoxaban for administering to a patient, the method comprising:
receiving simulated population data calculated using a plasma level prediction model; and
training a machine learning dosage calculator using the simulated population data.

The simulated population data may comprise simulated population data for at least 100,000 simulated patients.

The simulated population data may comprise simulated patient data, simulated dosages and/or simulated plasma level metrics.

The method may comprise calculating the simulated population data using the plasma level prediction model.

Calculating simulated population data using a plasma level prediction model may comprise:
defining simulated patient data for a patient population comprising a plurality of simulated patients and a corresponding plurality of simulated dosages; and
calculating plasma level metrics for each of the plurality of simulated patients by processing the simulated dosages and simulated patient data using the plasma level prediction model to determine simulated plasma level metrics;
determining the simulated population data comprising the simulated patient data, the simulated dosages and the simulated plasma level metrics.

The simulated patient data may comprise one or more of: a kidney function metric; a patient age; a patient ethnicity; a patient sex; a patient weight; a patient genotype; a patient cardiac metric; and a patient medication list.

Defining the simulated patient data may comprise: receiving a population distribution for each parameter type of the simulated patient data; and generating each simulated patient by probabilistic selection of each parameter type according to the respective population distribution. Defining the simulated patient data may comprise: defining a plurality of discrete values for each parameter type; and generating each simulated patient data as a different combination of one discrete value from each parameter type. Generating each simulated patient may comprise generating simulated patient data for every possible combination of one discrete value from each parameter type.

The plasma level prediction model may comprise a time-based differential equation model for modelling a time dependence of a plasma concentration of edoxaban as a function of the patient data.

The plasma level prediction model may comprise a compartment model for modelling a time dependence of a plasma concentration of edoxaban as a function of the patient data.

Training the machine learning dosage calculator may comprise training the machine learning dosage calculator using the simulated population data and real patient data.

The method may comprise validating the machine learning model using further simulated population data. The further simulated population data may be different to the simulated population data.

The method may comprise locking the machine learning dosage calculator to prevent further adjustment to the machine learning dosage calculator.

The method may further comprise calibrating the machine learning algorithm for a patient based on a measured drug plasma level or coagulation measure obtained from a blood test on the patient.

The machine learning dosage calculator may be configured to directly calculate the dosage for administering to the patient by processing patient data and a target plasma level metric.

The patient data may comprise one or more of: a kidney function metric; a patient age; a patient ethnicity; a patient gender; a patient weight; and a patient medication list.

According to a fourth aspect of the present disclosure there is provided a dosage calculator for determining a dosage of edoxaban for administering to a patient, the dosage calculator comprising one or more processors configured to:
receive patient data relating to a patient, wherein the patient data includes a kidney function metric;
process the patient data with a dosage calculator to determine the dosage of edoxaban for administering to the patient, wherein the dosage calculator is derived from a plasma level prediction model; and
indicate the dosage.

According to a fifth aspect of the present disclosure there is provided a computer implemented method for determining a procedure wait time for a patient following withdrawal of a direct oral anticoagulant, DOAC, in advance of an invasive procedure, the method comprising:
receiving patient data relating to a patient, wherein the patient data includes a kidney function metric and a DOAC dosage;
processing the patient data with a dosage calculator to determine the procedure wait time for a drug plasma level to fall below an invasive procedure plasma level threshold, wherein the dosage calculator is derived from a plasma level prediction model; and
indicating the procedure wait time.

The DOAC may comprise apixaban, dabigatran, rivaroxaban or edoxaban.

The method may comprise administering the procedure wait time in advance of the invasive procedure to reduce a risk of haemorrhage during the invasive procedure.

The patient data may further comprise one or more of: a patient age; a patient ethnicity; a patient sex; a patient weight; a patient genotype; a patient cardiac metric; and a patient medication list.

The patient genomic type may comprise a patient genotype for P-gp transporter genes such as ABCG or metabolic enzymes such as CYP 3A4/5.

The patient medication list may comprise an indication of whether the patient is consuming one or medications comprising: a proton-pump inhibitor; a calcium channel blocker; a P-gp inhibitor; an antifungal; an antiarrhythmic drug; an antibiotic; or a drug that increases a bleeding risk including a drug with an anticoagulant effect.

The one or more medications may comprise one or more of:
a proton pump inhibitors such as esomeprazole;
an antifungal such as ketoconazole;
an antiarrhythmic such as amiodarone or dronedarone;
an antibiotic such as rifampicin;
a drug that increases a bleeding risk such as a nonsteroidal anti-inflammatory drug, aspirin or clopidogrel.

According to a sixth aspect of the present disclosure, there is provided a method for administering a dosage of edoxaban to a patient for the treatment or prevention of thrombosis, the method comprising:
administering a dosage of edoxaban to the patient, wherein the dosage of edoxaban is determined by:
   receiving patient data relating to a patient, wherein the patient data includes a kidney function metric of the patient; and
   processing, using one or more processors, the patient data with a dosage calculator to determine the dosage of edoxaban for the patient, wherein the dosage calculator is derived from a plasma level prediction model that predicts edoxaban drug plasma levels, and the dosage calculator determines the dosage for the patient based in part on the kidney function metric of the patient.

According to a seventh aspect of the present disclosure there is provided edoxaban for use in the treatment of stroke prevention, thrombosis treatment or blood clot prevention, wherein the edoxaban dosage is determined by steps comprising:
receiving patient data relating to a patient, wherein the patient data includes a kidney function metric;
processing the patient data with a dosage calculator to determine the dosage of edoxaban for administering to the patient, wherein the dosage calculator is derived from a plasma level prediction model; and
indicating the dosage.

According to an eighth aspect of the present disclosure there is provided a method of treating thrombosis, stroke prevention or blood clot prevention comprising administering edoxaban to a patient in need thereof, wherein the edoxaban dosage is determined by the steps comprising:
receiving patient data relating to a patient, wherein the patient data includes a kidney function metric;
processing the patient data with a dosage calculator to determine the dosage of edoxaban for administering to the patient, wherein the dosage calculator is derived from a plasma level prediction model; and
indicating the dosage.

The patient data may further comprise one or more of: a patient age; a patient ethnicity; a patient sex; a patient weight; a patient genotype; a patient cardiac metric; and a patient medication list.

There may be provided a computer program, which when run on a computer, causes the computer to configure any apparatus, including a circuit, controller, converter, or device disclosed herein or perform any method disclosed herein. The computer program may be a software implementation, and the computer may be considered as any appropriate hardware, including a digital signal processor, a microcontroller, and an implementation in read only memory (ROM), erasable programmable read only memory (EPROM) or electronically erasable programmable read only memory (EEPROM), as non-limiting examples. The software may be an assembly program.

The computer program may be provided on a computer readable medium, which may be a physical computer readable medium such as a disc or a memory device, or may be embodied as a transient signal. Such a transient signal may be a network download, including an internet download. There may be provided one or more non-transitory computer-readable storage media storing computer-executable instructions that, when executed by a computing system, causes the computing system to perform any method disclosed herein.

### BRIEF DESCRIPTION OF THE DRAWINGS

One or more embodiments will now be described by way of example only with reference to the accompanying drawings in which:

FIG. 1A shows relationships between plasma edoxaban levels and anti-factor Xa activity and prothrombin time;

FIG. 1B illustrates an edoxaban exposure-response relationship for all strokes and systemic embolic events within 1 year in patients with NVAF;

FIG. 1C illustrates an edoxaban exposure-response relationships for major bleeds in patients with NVAF;

FIG. 2 illustrates a method for determining a dosage of edoxaban for administering to the patient according to an embodiment of the present disclosure;

FIG. 3 illustrates an example two-compartment model that may provide the plasma level prediction model according to one or more embodiments;

FIG. 4A illustrates the edoxaban plasma levels at steady state for a patient taking 60 mg edoxaban once daily calculated by a plasma level prediction model according to embodiments of the present disclosure;

FIG. 4B illustrates a relationship between renal function and edoxaban plasma levels for a patient taking 60 mg edoxaban once daily, as determined by a plasma level prediction model according to an embodiment of the disclosure;

FIG. 4C illustrates the relationship between body weight and edoxaban plasma levels for a patient taking 60 mg edoxaban once daily, as determined by a plasma level prediction model according to an embodiment of the disclosure;

FIG. 4D illustrates a relationship between P-glycoprotein inhibition and edoxaban plasma levels for a patient taking 60 mg edoxaban once daily, as determined by a plasma level prediction model according to an embodiment of the disclosure;

FIG. 4E illustrates the relationship between the presence/absence of non-valvular atrial fibrillation and edoxaban plasma levels for a patient taking 60 mg edoxaban once daily, as determined by a plasma level prediction model according to an embodiment of the disclosure;

FIG. 4F illustrates a relationship between ethnicity (Asian vs non-Asian) and edoxaban plasma levels for a patient taking 60 mg edoxaban once daily, as determined by a plasma level prediction model according to an embodiment of the disclosure;

FIG. 4G illustrates the effect of inter-subject variability on the 24-hour plasma concentration levels of edoxaban at steady state for a patient taking 60 mg edoxaban once daily, as determined by a plasma level prediction model according to an embodiment of the disclosure.

FIG. 5 illustrates a model of determining a dosage for administering to a patient according to an embodiment of the present disclosure;

FIG. 6 illustrates a method of generating a machine learning dosage calculator according to an embodiment of the present disclosure;

FIG. 7 illustrates a sub-sample of simulated population data;

FIG. 8A illustrates evaluation results of a dense neural network-based machine learning regression dosage calculator according to an embodiment of the present disclosure;

FIG. 8B illustrates evaluation results of a dense neural network-based machine learning classification dosage calculator according to an embodiment of the present disclosure;

FIG. 9 illustrates evaluation results of a dense neural network-based machine learning dosage calculator according to another embodiment of the present disclosure;

FIG. 10 illustrates a system suitable for carrying out any of the computer implemented methods described herein;

FIG. 11 illustrates estimated plasma level time profiles at steady state for a simulated patient on a recommended dose of 60 mg once daily, a novel dose of 30 mg twice daily and a novel dose of 90 mg once daily, calculated by a plasma level prediction model according to an embodiment of the present disclosure;

FIG. 12 illustrates an example patient monitoring process according to an embodiment of the present disclosure;

FIGS. 13A and 13B schematically illustrate the determination of procedure wait times for an example patient according to embodiments of the present disclosure;

FIG. 14 plots the predicted procedure wait time calculated with a machine learning dosage calculator versus the predicted procedure wait time calculated with a plasma level prediction model according to an embodiment of the present disclosure;

FIG. 15 illustrates a method for administering a dosage of edoxaban to a patient for the treatment or prevention of thrombosis according to an embodiment of the present disclosure; and

FIG. 16 illustrates a method for determining and administering a procedure wait time for a patient following withdrawal of a direct oral anti-coagulant in advance of an invasive procedure according to an embodiment of the present disclosure.

### DETAILED DESCRIPTION

### Edoxaban Pharmacokinetics (Pk)—Plasma Level Variability

Absorption is relatively rapid with peak plasma concentrations of the drug occurring after 1-2 hr following oral administration with a bioavailability of approximately 62% [3] due to P-glycoprotein (P-gp) elimination with ˜30-50% of unchanged drug found in the faeces partly due to biliary elimination. Food does not affect the total exposure but decreases the rate of absorption. The pharmacokinetics appears to be linear over the therapeutic range of doses. Plasma protein binding is relatively low (55%) and the drug is distributed in the body with a volume of distribution of 107 L (Parasrampuria & Truitt, 2016). Metabolism is not extensive (<30%) and occurs by carboxylesterase 1 (CES1), cytochrome P450 3A4 (CYP3A4), and via glucuronidation. The terminal half-life has been found to range from 10-14 hr with minimal accumulation upon repeat dosing. Edoxaban is eliminated primarily as unchanged drug in the urine. Renal clearance accounts for ˜50% of total body clearance. Total systemic exposure (AUC_{∞}) to edoxaban increased almost two-fold in subjects with moderate or severe renal impairment compared to subjects with normal renal function [1]. Edoxaban is a substrate of P-gp, while its active metabolite, D21-2393, is a substrate of the uptake transporter OATP1B1, and thus multiple drug interactions are possible.

Because of the large effect of renal function on edoxaban PK and increased bleeding, guidelines in the USA for use of edoxaban are complex and exclude patients that might benefit from edoxaban if prescribed and monitored in a more sophisticated way. The guidelines are:

### Treatment of NVAF

Renal clearance (CrCl) should be assessed before initiating therapy. The recommended dose is 60 mg once daily in patients with CRCL between 50 and 95 mL/min. Edoxaban should not be used when CRCL>95 mL/min. The dose should be reduced to 30 mg once daily in patients with CrCL between 15 and 50 mL/min.

### Treatment of DVT and PE

The recommended dose is 60 mg once daily. However, for patients with CrCl between 15 and 50 mL/min, body weight less than or equal to 60 kg, or those who use certain P-gp inhibitors, the recommended dose is 30 mg once daily.

However, there is still large inter-subject variability in the resulting measures of coagulation and outcomes between underdosed (increased morbidity and mortality) and overdosed (increased haemorrhagic incidences and GI disturbances).

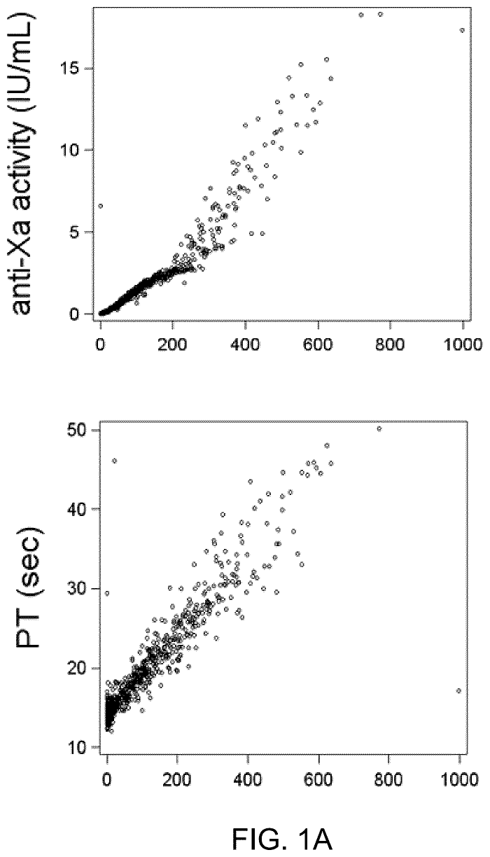

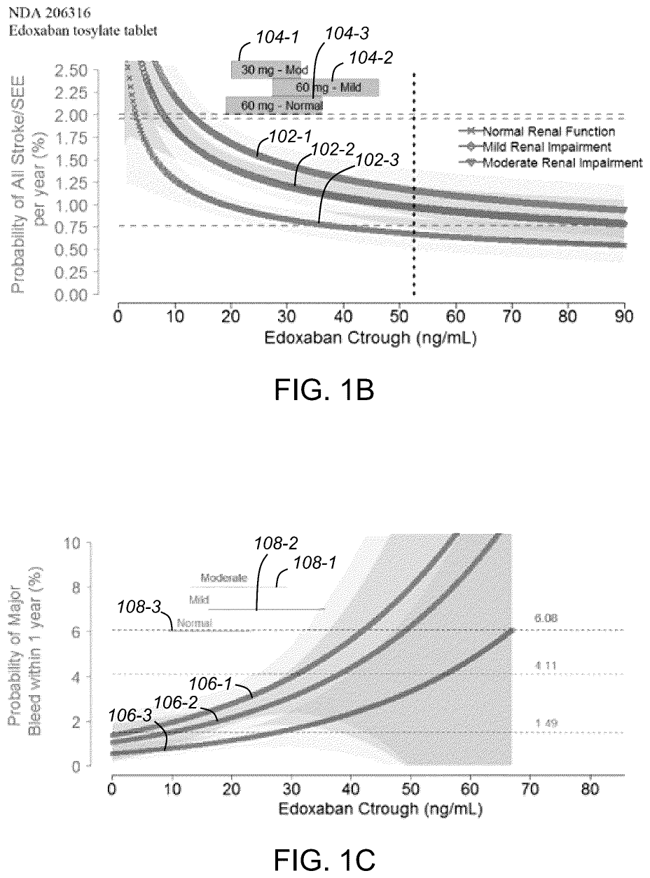

A trough level is the plasma level before a next scheduled dose (typically the morning dose). Several studies have shown relationships between plasma drug levels and clinical outcomes, suggesting that trough levels between 10-40 ng/ml provide the best balance between reduction in the risks of stroke and bleeding [2]. However, measuring drug levels in general practice is costly and is infrequently undertaken. FIG. 1A (taken from [1], [3]) shows substantially linear relationships between plasma edoxaban levels and particular measures of the activity of edoxaban on coagulation, namely anti-factor Xa (anti-FXa) activity and prothrombin time (PT) and these may form a way of monitoring patient outcomes, as discussed further below in the section ‘Ongoing Treatment Management.’

In summary, a large variability in drug plasma levels between patients is driven by: the influence of renal function; the effect of other drugs; low absorption; and other factors (discussed further below). FIG. 1B and FIG. 1C (taken from [2]) illustrate relationships between edoxaban plasma levels and the probability of clinical outcomes.

FIG. 1B illustrates the exposure-response relationship for all strokes and systemic embolic events within 1 year in patients with NVAF, by renal function. The Figure illustrates ranges of trough plasma levels (5th to 95th percentiles) measured for a population of patients. A first range 104-1 corresponds to a first population of patients with moderate renal impairment receiving 30 mg edoxaban. A second range 104-2 corresponds to a second population of patients with mild renal impairment receiving 60 mg edoxaban. A third range 104-3 corresponds to a third population of patients with normal renal function receiving 60 mg edoxaban. A first line plot 102-1 illustrates the variation in probability of stroke/systemic embolic events with Edoxaban trough concentration for the first population of patients. A second line plot 102-4 1 illustrates the variation in probability of stroke/systemic embolic events with Edoxaban trough concentration for the second population of patients. A third line plot 102-3 1 illustrates the variation in probability of stroke/systemic embolic events with Edoxaban trough concentration for the third population of patients. The plots 102 show that the risk of stroke or systemic embolic event (SEE) decreases with increasing edoxaban trough levels in patients with NVAF. To reduce the risk of a stroke or SEE below 1.25%, a patient with normal renal function requires a trough edoxaban level above approximately 10 ng/ml. To reduce the risk of a stroke or SEE below 1%, a patient with normal renal function requires a trough edoxaban level above approximately 20 ng/ml.

FIG. 1C illustrates exposure-response relationships for major bleeds in patients with NVAF, by renal function. The figure also illustrates ranges of trough plasma levels (5th to 95th percentiles) measured for a population of patients. Similar to FIG. 1B, a first range 108-1 corresponds to a first population of patients with moderate renal impairment receiving 30 mg edoxaban. A second range 108-2 corresponds to a second population of patients with mild renal impairment receiving 60 mg edoxaban. A third range 104-3 corresponds to a third population of patients with normal renal function receiving 60 mg edoxaban. A first line plot 106-1 illustrates the variation in probability of major bleed events with Edoxaban trough concentration for the first population of patients. A second line plot 102-4 1 illustrates the variation in probability of major bleed events with Edoxaban trough concentration for the second population of patients. A third line plot 102-3 1 illustrates the variation in probability of major bleed events with Edoxaban trough concentration for the third population of patients. The Figure shows that the risk of a major bleeding event increases with increasing edoxaban trough levels in patients with NVAF. To reduce the risk of a major bleeding event below 2.5%, a patient with normal renal function requires a trough edoxaban level below approximately 40 ng/ml. To reduce the risk of a major bleeding event below 2%, a patient with normal renal function requires a trough edoxaban level below approximately 30 ng/ml.

Therefore, from FIGS. 1B and 1C, it can be seen that for a person with normal renal function, the best balance between reducing the risks of stroke and bleeding involves trough levels between 10 and 40 ng/ml. The centre of this range constitutes an ideal therapeutic level (ITL) of ˜25 ng/ml.

### OVERVIEW OF THE INVENTION

The present disclosure provides a method for determining a dosage of edoxaban for administering to a patient that utilizes a dosage calculator derived from a plasma level prediction model. The method may determine the dosage without requiring plasma drug level measurements. The dosage calculator can process patient data including a kidney function metric to indicate a dosage for administering to the patient.

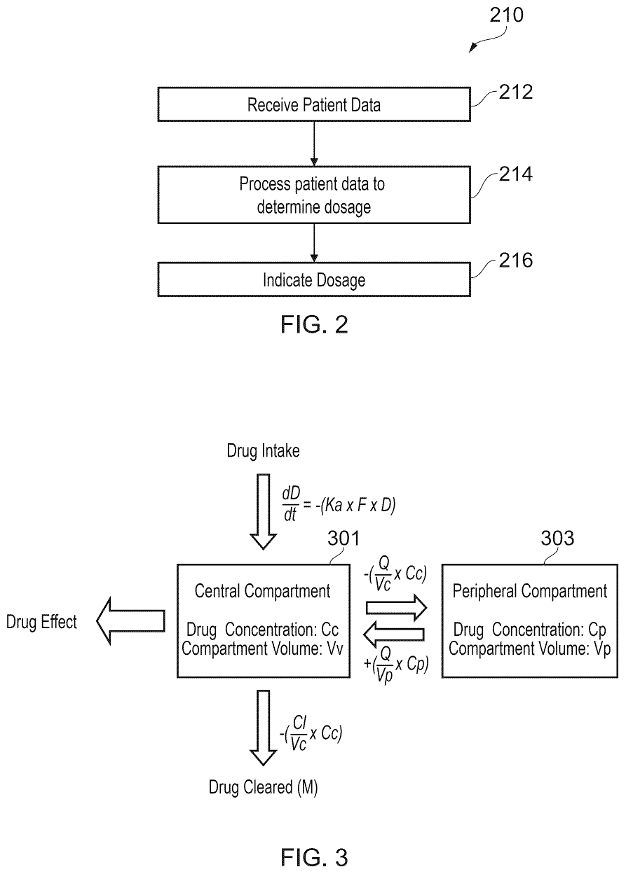

FIG. 2 illustrates a method 210 for determining a dosage of edoxaban for administering to the patient according to an embodiment of the present disclosure.

A first step 212 comprises receiving patient data relating to a patient. The patient data includes a kidney function metric, which may be a measured creatinine clearance. In some examples the kidney function metric may comprise other renal function metrics such as Inulin cystatin C, beta trace proteins, 51Cr-EDTA (radioactive chromium complexed with ethylenediaminetetraacetic acid), 99Tc-EDTA (radioactive technetium complexed with ethylenediaminetetraacetic acid). As described above and below, kidney function is the most significant driver of edoxaban drug plasma level variability between patients.

A second step 214 comprises processing the patient data with a dosage calculator to determine the dosage of edoxaban for administering to the patient. The dosage calculator is derived from a plasma level prediction model. Example dosage calculators and plasma level prediction models are discussed in detail below.

A third step 216 comprises indicating the dosage.

The method advantageously accounts for the biggest driver of variability in edoxaban drug plasma levels—kidney function. The term edoxaban drug plasma level may also be referred to herein as edoxaban blood plasma level, blood plasma level, drug plasma level or simply plasma level. The term level may be referred to as a concentration. As described below, embodiments of the method can process patient data comprising a plurality of parameters that contribute to plasma level variability, such as demographic data, medication data and patient condition data, to advantageously provide more accurate dosages for patients. The method can calculate a starting dose for a patient.

The method can also continue to monitor patient data and provide a revised dose for a patient as their patient data changes. In this way, the method can address the provision of inaccurate starting doses and the lack of ongoing monitoring for DOACs described above. As described below, the method may indicate dosage amounts that differ from the standard dosages available. In some embodiments, the method may recommend novel dosage amounts provided by novel dosage forms including liquid or microgranule formulations or other known solubility enhancement formulations. As mentioned above, microgranules may improve absorption which may reduce inter-subject variability in drug plasma levels. This can advantageously make edoxaban available for patients for which the drug is currently contra-indicated and provide precision dosing for patients in which achieving the correct plasma level is critical (e.g. cancer patients). As described below, the method can also encompass personalisation by providing personalised target plasma metrics on an individual basis based on the patient data.

### Plasma Level Prediction Model

In relation to the second step 214, the plasma level prediction model may comprise a theoretical model that can predict an edoxaban drug plasma level based on a dosage and the patient data. In some examples, the plasma level prediction model may be a time-dependent differential equation-based (or rate equation-based) model. The plasma level prediction model may comprise a compartment model such as a two-compartment model, or any other PK-PD analytical model for predicting the drug plasma level as a function of patient data.

### Description of a Two-Compartment PK Model

FIG. 3 illustrates an example two-compartment (or dual-compartment) model that may provide the plasma level prediction model according to one or more embodiments. A central compartment 301 may represent a target location in the body in which the drug provides a therapeutic effect. In the example of edoxaban, the central compartment 301 represents the blood or plasma, however the central compartment 301 may comprise other locations (e.g. liver, tissue, etc) for other drugs. The central compartment 301 has a central compartment drug concentration, Cc, (the drug plasma level in this example) and a central compartment volume, Vc. Drug intake to the central compartment 301 from the administered drug may be defined by:

$$\frac{d D}{d t} = - ( K a \times F \times D ) \qquad ( Equation 1 )$$

where Ka is a first order drug absorption rate constant: F is a relative bioavailability of the drug; and D is the drug dosage.

Drug clearance from the central compartment 301, for example via the kidneys, may be defined as:

$$\frac{d {C}_{C L}}{d t} = + ( \frac{C l}{V c} \times C c ) \qquad ( Equation 2 )$$

Where C_{CL} is the concentration of drug cleared from the central compartment, for example via the kidneys, Cc is the concentration of drug in the central compartment, and CL is the total body clearance of the drug from the central compartment.

A peripheral compartment 303 may represent locations in the body in which the drug may reside and not necessarily provide a therapeutic effect. The peripheral compartment 303 has a peripheral compartment drug concentration, Cp, and a peripheral compartment volume, Vp. A rate of change of the peripheral compartment drug concentration, Cp, resulting from the flow of drug between the two compartments may be defined as:

$$\frac{d C p}{d t} = + ( \frac{Q}{V c} \times C c ) - ( \frac{Q}{V p} \times C p ) \qquad ( Equation 3 )$$

where Q is an intercompartmental clearance between the central and peripheral compartments.

Combining everything together, the rate of change of the central compartment drug concentration, Cc, can be written as:

$$\frac{d C c}{d t} = + ( \frac{K a}{V c} \times F \times D ) - ( \frac{C l}{V c} \times C c ) - ( \frac{Q}{V c} \times C c ) + ( \frac{Q}{V p} \times C p ) \qquad ( Equation 4 )$$

Estimated values of the specific parameters for the model can be obtained from a patient study.

Table 1 shows the population mean values of various PK parameters from a selection of edoxaban pop-PK models, which are all examples of plasma level prediction models. The table also shows the covariates that were found to have a significant influence on each PK parameter.

TABLE 1
Comparison of PK parameters and significant
covariates from various pop-PK studies.

| | | | | Niebecker | Krekels |
|---|---|---|---|---|---|
| | Salazar et | Song et al. | Yin et al. | et al. | et al. |
| | al. (2012) | (2014) | (2014) | (2015) | (2016) |
| Paper | [4] | [5] | [6] | [7] | [8] |
| Types of study | Phases I | Phases I | Phases I, | Phases I | Phases I |
| involved | and II | and II | II and III | and III | and III |
| CL/F (L/h) | 36 | 32.1 | 11.4 | 15.5 | 13.8 |
| | | | | (CL_{NR}/F) | (CL_{NR}/F) |
| Vc/F (L) | 244 | 214 | 151 | 209 | 194 |
| Vp/F (L) | 90.3 | 134 | 42.9 | 92.3 | 88.6 |
| Q/F (L/hr) | 6.42 | 8.85 | 2.73 | 5.92 | 5.75 |
| Ka (hr⁻¹) | 5.87 | 3.54 | 1.08 | 3.35 | 2.16 |
| Lag time (hr) | 0.42 | 0.39 | 0.25 | 0.25 | 0.25 |
| F | 1 | | 0.58 | | |
| Significant | | | | | |
| covariates | | | | | |
| Age | NS | NE | CL/F, Ka | NS | NS |
| Body weight | V_{c}/F | NE | CL/F, V_{c}/F | CL_{NR}/F, Q/F, | CL_{NR}/F, Q/F, |
| | | | | V_{c}/F, Vₚ/F | V_{c}/F, Vₚ/F |
| Sex | NS | NE | NS | NS | NS |
| CRCL | CL/F | NE | CL/F | CL/F | CL/F |
| Race | NS | NE | NS | V_{c}/F | CL/F, |
| | | | | | Vₚ/F, F |
| Patient status | V_{c}/F | NE | NE | NE | CL/F, F, |
| | | | | | Q/F |
| Food | Ka | NE | NE | NE | NS |
| P-gp | CL/F, Ka, F | NE | V_{c}/F, Ka, F | CL/F, F | F |
| inhibition | | | | | |

CL_{NR} = non-renal clearance; NE = not examined; NS = not significant.

The weaker effect of P-gp inhibitors found in phase III studies (16%) compared to phase II studies (120%) could be because a) of the less well-controlled environment of a phase III study, b) less than 5% of the PK observations involved patients taking P-gp inhibitors c) the phase III studies included generally less potent P-gp inhibitors compared to the phase II studies. Thus guidance on whether to use dose reduction should be on a drug-by-drug basis. For strong P-gp inhibitors, the US prescribing information recommends halving the edoxaban dose for the treatment of DVT and PE.

All studies have used, to some extent, data from phase I studies as a baseline, and then showed differences depending on which patients were examined. All basic PK parameters are similar across all studies except for clearance in Salazar and Song studies, where clearance was faster in phase I/II healthy volunteers, possibly reflecting slower metabolic rate, poorer absorption or lack of compliance in phase III subjects. Clearance is mostly affected by renal clearance, and to a lesser extent by P-gp inhibitors (particularly in phase III studies), age, body weight, and gender. Volume is affected by body weight and if the patient is of Asian origin, which may also be influenced by body weight. Edoxaban bioavailability, F, is affected by P-gp inhibitors, age, and patient status, while food intake delays the tₘₐₓ.

Taking a closer look at the model of Krekels et al. (2016), the model comprises a two-compartment population PK model for edoxaban. Their model was based on data from 443 healthy volunteers from 13 phase I studies and 10,432 patients with non-valvular atrial fibrillation (NVAF) from the ENGAGE AF-TIMI 48 phase III study. The studies involved edoxaban doses ranging from 15 to 60 mg. Various covariates (patient data) thought to relate to the drug's PK were collected, such as age, sex, ethnicity, presence of NVAF, body weight, creatinine clearance, and co-administration with known P-gp inhibitors, such as dronedarone, erythromycin, ketoconazole, quinidine, and verapamil. Apparent renal clearance was influenced by creatinine clearance and the presence of NVAF. Apparent non-renal clearance was influenced by body weight and the presence of NVAF. Once renal and non-renal components were combined, apparent clearance was influenced by ethnicity (Asian vs non-Asian), presence of NVAF, and co-administration with P-gp inhibitors. Apparent central compartment volume and apparent intercompartmental clearance were both influenced by ethnicity (Asian vs non-Asian) and body weight. Apparent peripheral compartment volume was influenced by body weight. Finally, bioavailability was influenced by ethnicity (Asian vs non-Asian), presence of NVAF, and co-administration with P-gp inhibitors.

The following modifiers for apparent clearance (renal, non-renal, and combined), apparent central compartment volume, apparent peripheral compartment volume, apparent intercompartmental clearance, and bioavailability were as follows (not including the variability components):

$${Slope}_{i} = 0.0503 \cdot ( 1 - {NVAF}_{i} ) - 0.0503 \cdot 0.851 \cdot {NVAF}_{i} \qquad ( Equation 5 )$$

$$C {L}_{R} / {F}_{i} = 0 \text{.197} \cdot {CRCL}_{i} [ if {CRCL}_{i} \leq 90 ml / min ] \qquad ( Equation 6 )$$

$${CL}_{R} / {F}_{i} = 90 ( 0.197 - {Slope}_{i} ) + {Slope}_{i} \cdot {CRCL}_{i} > [ if {CRCL}_{i} 90 ml / min ] \qquad ( Equation 7 )$$

$${CL}_{N R} / {F}_{i} = 13.8 \cdot ( 1 \cdot ( 1 - {NVAF}_{i} ) + 0.851 \cdot {NVAF}_{i} ) \cdot {( \frac{{weight}_{i}}{70} )}^{0.75} \qquad ( Equation 8 )$$

$$CL / {F}_{i} = ( {CL}_{R} / {F}_{i} + {CL}_{NR} / {F}_{i} ) \cdot ( 1 + 0.316 \cdot ( 1 - {NVAF}_{i} ) \cdot {Pgp}_{i} ) \cdot ( 1 + 0.48 \cdot {Asian}_{i} ) \cdot {e}^{{η}_{CL / F}} \qquad Equation 9$$

$${V}_{c} / {F}_{i} = 194 \cdot ( 1 + 0.74 \cdot {Asian}_{i} ) \cdot {( \frac{{weight}_{i}}{70} )}^{0.75} \cdot {e}^{( 1.58 \cdot {η}_{{V}_{c} / F} )} \qquad ( Equation 10 )$$

$${V}_{p} / {F}_{i} = 8 \; 8.6 \cdot {( \frac{{weight}_{i}}{70} )}^{0.75} \cdot {e}^{{η}_{{V}_{p} / F}} \qquad ( Equation 11 )$$

$$Q / {F}_{i} = 5.75 \cdot ( 1 + 2.98 \cdot {NVAF}_{i} ) \cdot {( \frac{{weight}_{i}}{70} )}^{0.75} \cdot {e}^{{η}_{Q / F}} \qquad ( Equation 12 )$$

$${F}_{i} = ( 1 + 1.2 \cdot 0.134 \cdot {Pgp}_{i} \cdot {NVAF}_{i} ) \cdot ( 1 + 1.2 \cdot {Pgp}_{i} \cdot ( 1 - {NVAF}_{i} ) ) \cdot ( 1 + 0.417 \cdot {Asian}_{i} ) \qquad Equation 13$$

Here, CL_{R}/Fᵢ and CL_{NR}/Fᵢ are the apparent renal and non-renal clearances for an individual, respectively, CL/Fᵢ is the apparent clearance for an individual, V_{c}/Fᵢ is the apparent central compartment volume for an individual, Vₚ/Fᵢ is the apparent peripheral compartment volume for an individual, Q/Fᵢ is the apparent intercompartmental clearance for an individual, and Fᵢ is the bioavailability for an individual. Similarly, CRCLᵢ and weightᵢ are the creatinine clearance and body weight for an individual, respectively, while Asianᵢ, NVAFᵢ, and Pgpᵢ are binary variables which take a value 1 when the individual is Asian, has NVAF, or is concomitantly taking a P-gp inhibitor, respectively, and a value 0 otherwise. η_{CL/F}, η_{Vc/F}, η_{Vp/F}, and η_{Q/F} are normally distributed variables with mean 0 and variance 0.018496, 0.046225, 0.228484, and 0.228484, respectively, and are included to model the between-subject variability.

According to one or more embodiments of the present disclosure, a two-compartment plasma level prediction model may be defined by equations 1 to 4, with expressions for apparent clearance, apparent central compartment volume, apparent peripheral compartment volume, apparent intercompartmental clearance, absorption rate constant, and bioavailability, adjusted according to equations 5 to 13 from Krekels et al. (2016) [8]. However, it will be appreciated that while a replica of Krekel's model is used here as an example of a plasma level prediction model, other plasma level prediction models are available or may be developed in future that fall within the scope of the present disclosure.

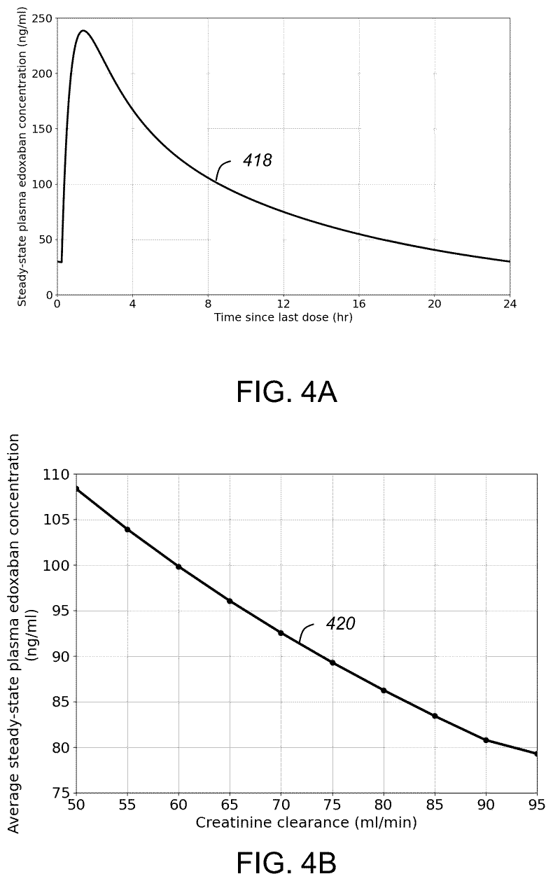

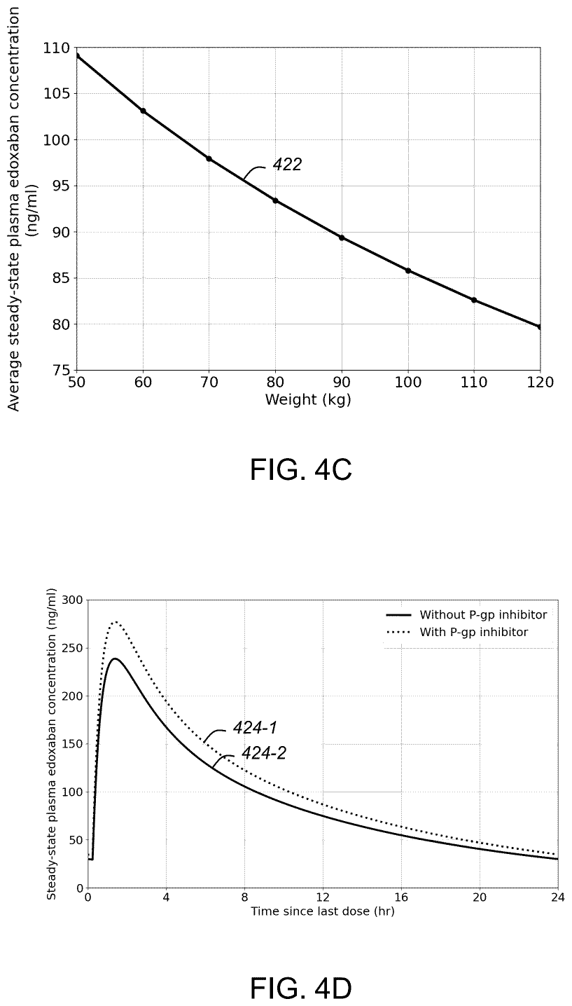

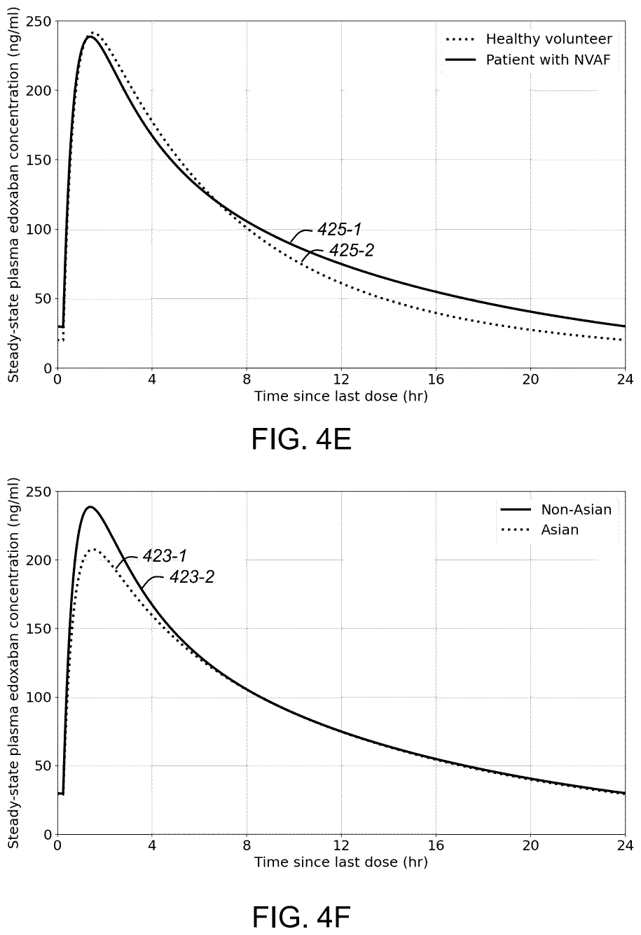

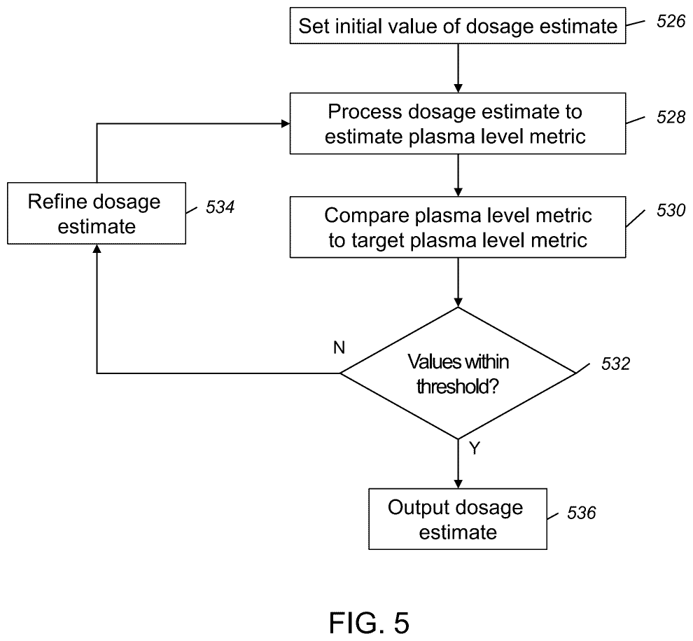

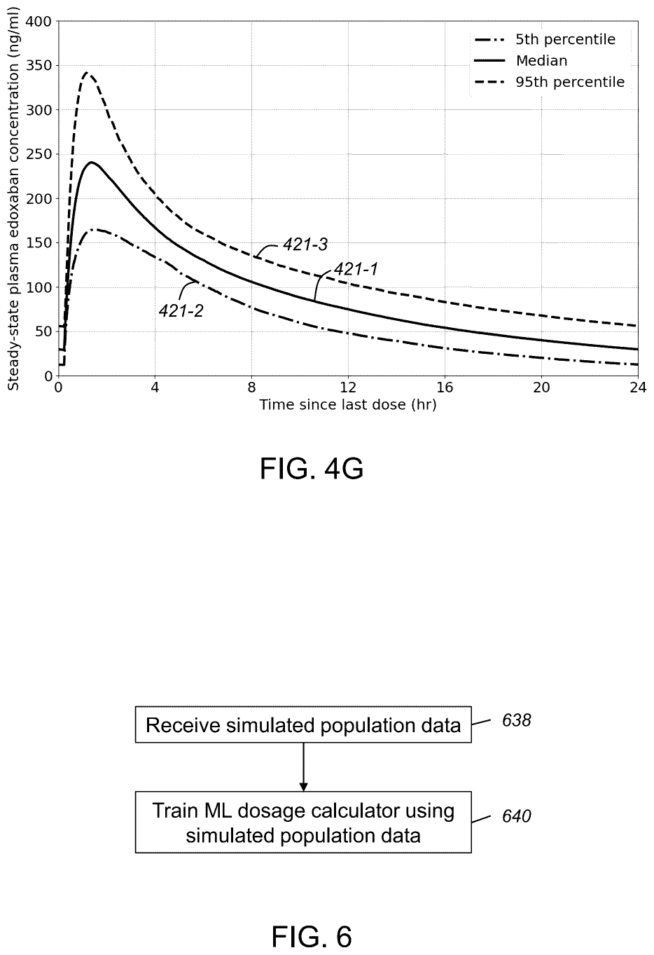

FIGS. 4A to 4G illustrate example plasma level predictions of a plasma level prediction model comprising the replica Krekel model. FIGS. 4A to 4F illustrate predictions with the normally distributed variables set to zero (mean=0 and variance=0). FIG. 4G illustrates the effect of the normally distributed variables which reflect inter-subject variability arising from factors other than those defined in the model. Each figure illustrates simulated data for a patient taking 60 mg of edoxaban once daily, the patient having non-valvular AF, a creatinine clearance of 70 ml/min, 82 kg weight, non-Asian ethnicity and not taking P-gp inhibitors.

FIG. 4A illustrates 24-hour plasma concentration levels 418 of edoxaban at steady state for the patient.

FIG. 4B illustrates the relationship 420 between renal function (CrCl) and average steady state plasma levels for the patient with the above characteristics (other than the dependent variable of renal function). The curve 420 illustrates that relatively small changes in CrCl can result in relatively large variations in circulating drug plasma levels.

FIG. 4C illustrates the relationship 422 between weight and average steady state plasma levels for the patient with the above characteristics (other than the dependent variable of weight). The plot illustrates the decreasing levels of circulating drug with increased body weight.

FIG. 4D illustrates the effect of comedication with a mild P-gp inhibitor on average steady state edoxaban plasma levels for the patient with the above characteristics (other than the dependent variable of P-gp comedication). A first plot 424-1 illustrates the patient plasma levels when comedicating with the P-gp inhibitor and a second plot 424-2 illustrates the patient plasma levels when not taking a P-gp inhibitor. The figure illustrates the increase in edoxaban plasma levels when taking a P-gp inhibitor.

FIG. 4E illustrates the effect of non-valvular AF on average steady state edoxaban plasma levels for the patient with the above characteristics (other than the dependent variable of NVAF). A first plot 425-1 illustrates the patient plasma levels when the patient has NVAF and a second plot 425-2 illustrates the patient plasma levels when the patient does not have NVAF. The figure illustrates the increase in edoxaban trough plasma levels for patients suffering from NVAF.

FIG. 4F illustrates the effect of Asian ethnicity on average steady state edoxaban plasma levels for the patient with the above characteristics (other than the dependent variable of ethnicity). A first plot 423-1 illustrates the patient plasma levels when the patient is of Asian ethnicity and a second plot 425-2 illustrates the patient plasma levels when the patient is not of Asian ethnicity. The figure illustrates the decrease in edoxaban peak plasma levels for patients of Asian ethnicity.

FIG. 4G illustrates the effect of inter-subject (between-subject) variability (arising from factors other than those defined in the model) on the 24-hour plasma concentration levels of edoxaban at steady state for the patient with the above characteristics. The inter-subject variability is modelled via normally distributed variables η_{CL/F}, η_{Vc/F}, η_{Vp/F}, and η_{Q/F} implemented in equations 9 to 12 and described above. The data for FIG. 4G was generated by simulating the 24-hour plasma concentration profile for the patient 1,000 times with each simulation including a random value for each of the normally distributed variables of equations 9 to 12 (here, random value refers to a random selection from the corresponding normal distribution profile). A first plot 421-1 illustrates the 50^{th} percentile 24-hour plasma concentration profile from the 1,000 simulations and substantially corresponds to the profile of FIG. 4A for which the normally distributed variables were set to zero. Second plot 421-2 and third plot 421-3 respectively correspond to the 5th and 95th 24-hour plasma concentration profile from the 1,000 simulations. The figure illustrates edoxaban plasma level variability of the PK parameters (e.g. CL, Vc, Vp, Q) which is not explained by covariates (weight, CRCL, ethnicity etc) defined in equations 1-12. For simplicity, variability is not included in any other disclosed figures so that the central tendencies of the curves can be clearly visualised.

It can be seen from the above equations for the model and from FIGS. 4A to 4F that although renal function, body weight, and co-administration with P-gp inhibitors are the main parameters used by regulatory agencies to dictate starting doses, there are other factors such as ethnicity that could be taken into account when computing plasma levels for a particular patient. This is particularly true when single or multiple extremes are taken into account, such as for a subject who is 50 kg, has a CRCL of 82 ml/min, and is taking P-gp inhibitor at the same time, where levels could be 45% higher than for a patient with “normal” (mean) values. As discussed below, the provision of a dosage calculator based on a plasma level prediction model can enable: (i) the provision of accurate and precise dosages; (ii) the provision of personalised dosages; (iii) the utilisation of a broad range of patient data, including data never before considered in pharmacometrics modelling; (iv) the provision of dosage monitoring and adjustment; (v) the definition of novel dosage regimens; and (vi) the provision of clot and bleeding risk according to time since last medication taken.

Embodiments of the present disclosure may comprise two-compartment models based on equations 1 to 4 and modified by other expressions for F, Vc/F, Vp/F, Q/F, and/or clearance derived from other patient studies of edoxaban, including studies conducted using the apparatus and methods disclosed herein. Other embodiments may include simpler single compartment models that do not include the peripheral compartment 303. However, two compartment models can provide more accurate results than single compartment models.

### Dosage Calculator

The plasma level prediction model can predict plasma levels throughout the day including peak or maximum plasma levels (Cₘₐₓ), average plasma levels at steady state (C_{average}) and trough levels (C_{trough}) based on the individual patient data. Returning to the method of FIG. 2, the method advantageously implements a dosage calculator derived from the plasma level prediction model enabling calculation of a starting dose (or dose adjustment) using Pharmacokinetic principles.

Returning to FIG. 2, in relation to the second step 214, the method processes the patient data with a dosage calculator derived from the plasma level prediction model. In some examples, the dosage calculator may receive a target plasma level metric and calculate the dosage for administering to the patient by processing the patient data and the target plasma level metric. The target plasma level metric may comprise an ideal therapeutic level (ITL) of the C_{trough} (e.g. 10-40 ng/ml or a more targeted level such as 25 ng/ml). In some examples the target plasma level metric may comprise an ITL of another plasma level metric such as Cₘₐₓ, C_{average}, AUC etc. The ITL is intended to provide the best balance between reducing the risk of thrombosis and embolism balanced against reducing the risk of haemorrhage. The ITL ranges and values disclosed herein may be illustrative of an optimum balance between thrombosis risk and bleeding risk at a population level. It will be appreciated that other ITL values may be used, such as future values established by parties of experts. Data from the disclosed systems and methods may inform, define or refine an ITL for edoxaban plasma concentration. Such ITL's may be defined for a plurality of patient groups with different risk factors based on personalised target plasma level metrics (See below). As noted above, the target plasma level metric may relate to other blood plasma metrics such as maximum concentration, average concentration, or area under the curve, and may be personalised based on the patient data, as discussed under the “Personalised Target Plasma Level Metrics” section below.

### Dosage Calculator Example 1

In some examples, the dosage calculator may determine a dose of edoxaban for administering to a patient using an analytical analysis. For example, the dosage calculator may determine the dosage based on pharmacokinetic principles and simple equations relating administered DOAC dose to Cₘₐₓ and C_{average}/AUC. In this way, a simple calculation can convert an ITL for C_{average} to an estimated dose. However, such models may not account for individual patient data (kidney function and other variables).

In some examples, analytical dosage calculators may be derived from single compartment plasma level prediction models that may be solved analytically to output a dosage for a particular target plasma level metric.

An advantage of this analytical approach is that iterative calculation of the differential equations (equations 1 to 4) of the plasma level prediction model are not required and this reduces the processing requirements required for the dosing calculation.

### Dosage Calculator Example 2

In some examples, the dosage calculator may implement a two-compartment plasma level prediction model utilising all of equations 1 to 13 of the above plasma level prediction model. The dosage calculator may then proceed according to the method of FIG. 5, which illustrates a method of determining a dosage for administering to a patient according to an embodiment of the present disclosure.

A first step 526 comprises setting an initial value of a dosage estimate.

A second step 528 comprises processing the dosage estimate with the dosage calculator to determine a plasma level metric. The plasma level metric may comprise a trough plasma level, Ctrough, a maximum plasma level, Cmax, a ratio of the maximum to trough plasma levels, an average plasma level over a dosing interval at steady state (i.e. once the drug has accumulated and stabilised over a number of days), C_{average}, or any other plasma level metric described herein.

A third step 530 comprises comparing the plasma level metric to the target plasma level metric. The target plasma level metric may comprise the same metric (trough level, Cmax, Caverage, etc) as the plasma level metric.

A fourth decision step 532 comprises determining if a difference in the values of the plasma level metric and the target plasma level metric is within a difference threshold. If not the method proceeds to a fifth step 534 and refines the dosage estimate, before returning to the second step 528.

If the difference in the two values is within the difference threshold, the method proceeds to a sixth step 536 and outputs or indicates the dosage estimate as the dosage for administering to a patient.

In some examples, the loop around the second to fifth steps 528-534 may be performed in an iterative fashion until the values are within the difference threshold. In some examples, the loop may be performed at least two times and a dosage value corresponding to the target plasma level metric may be interpolated. In some examples, the loop may correspond to an optimisation routine.

A disadvantage of dosage calculator example 2 is that the second step 528 requires a lot of processing power for calculating the blood plasma metric using the piecewise time-step differential equations above (equations 1 to 4). As explained in the “Implementations” section below, the method of FIG. 2 may be implemented as one or both of: (i) a software based dosage calculator configured to indicate dosages in a short timeframe (sub-seconds, seconds, or minutes) to a health practitioner and/or patient via a personal computing device or the like; and/or (ii) a software-based dosage calculator configured to determine a relational mapping between starting dosage, patient data and target plasma level metric. Such a relational mapping may take the form of a look-up table, a nomogram, an analogue computer or the like. Dosage calculator example 2 may be used in the second such implementation. For example, a detailed relational mapping can be determined over a long time frame (hours, days, months) by performing the method of FIG. 5 for a population set of different patient data. The resulting output can provide a detailed mapping between target plasma level metric (e.g. ITL) and starting dose for a range of the key patient data variables (e.g. kidney function, age, ethnicity, sex, medications etc). The relational mapping (e.g. look-up tables) can provide one or more of: (i) a dosage calculator for the first implementation of the method of FIG. 2 (i.e. quick dosage calculation for patient/HCP); (ii) a training data set for a ML dosage calculator (see Dosage Calculator Example 4); (iii) the foundations of a clinical guidelines reference; and (iv) standard dosing regimens for edoxaban products, regulatory labelling, packaging etc.

### Dosage Calculator Example 3

A third example dosage calculator may comprise a look-up table derived from the second dosage calculator example. The second step 528 of FIG. 5 can advantageously be performed rapidly using a look-up table approach. In some examples, the look-up tables may be defined to output the dosage for administering to the patient based on received inputs of the target plasma level metric and the patient data, negating the requirement for the process of FIG. 5 and resulting in more rapid dosage calculations.

### Dosage Calculator Example 4

In some examples, the dosage calculator may comprise a machine learning (ML) model (also referred to as a ML algorithm) trained using data output from the plasma level prediction model. The data output from the plasma level prediction model may comprise simulated population data. The simulated population data may comprise data output from the plasma level prediction model following processing of a population set of patient data that represents a population variation in the patient data. For example, a Monte Carlo type approach may be used to generate the population set of patient data using known distributions of each parameter type of the patient data (see below).

In some examples, the simulated population data may comprise plasma level metrics output by the plasma level prediction model. In some examples, the simulated population data may comprise dosages for administering to each patient of the population set to achieve a target plasma level. The ML model can be trained using the simulated population data to provide a ML plasma level prediction model and/or a ML dosage calculator.

FIG. 6 illustrates a method of generating a ML dosage calculator (or ML plasma level prediction model) according to an embodiment of the present disclosure.

A first step 638 comprises receiving simulated population data calculated using a plasma level prediction model.

A second step 640 comprises training the ML dosage calculator using the simulated population data.

The method may comprise calculating the simulated population data using the plasma level prediction model. For example, the method may comprise: (i) defining simulated patient data for a simulated patient population comprising a plurality of simulated patients; and (ii) calculating plasma level metrics for each of the plurality of simulated patients using the plasma level prediction model to define the simulated population data. The simulated population data may comprise the simulated patient data and the calculated plasma level metrics.

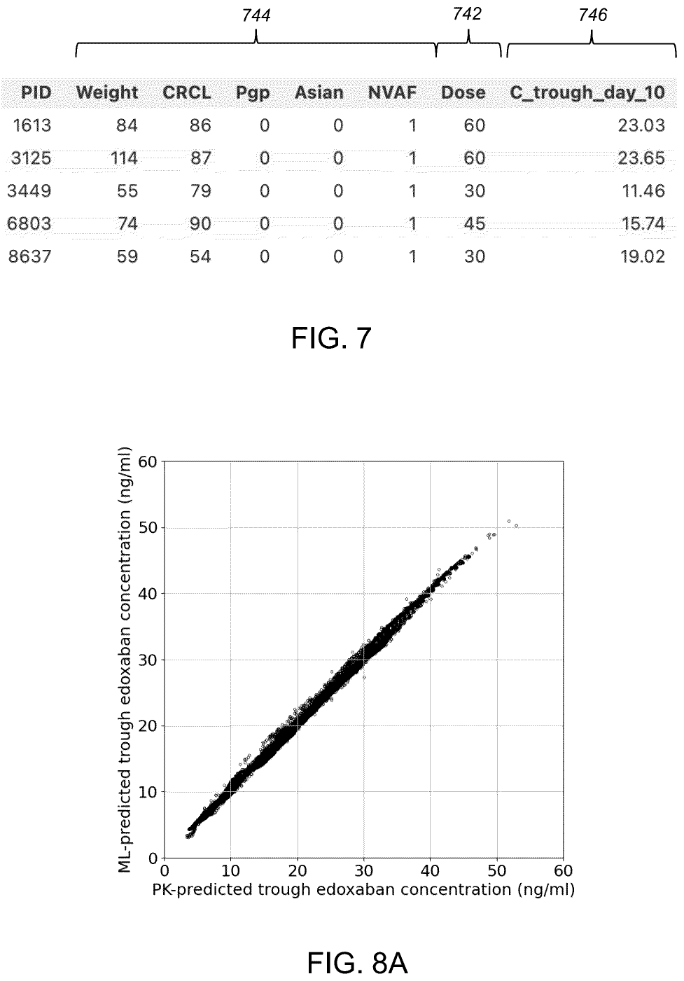

The simulated patient population may comprise at least 100,000 simulated patients, at least 1,000,000 simulated patients or at least 10,000,000 simulated patients. Defining the simulated patient data may comprise: (i) receiving population distributions for each parameter type of patient data (e.g. CrCL, ethnicity, weight, other medications, etc); and (ii) generating the plurality of simulated patients by probabilistic based selection of each parameter type according to the respective population distribution. For example, a Monte Carlo type analysis may be used to define the simulated patient data. In other examples, the simulated patient data may comprise a more methodical sweep of the parameter space defined by the patient data (FIG. 7). In some examples, the simulated patient data may also include a simulated dosage amount given twice daily. The simulated patient data (including the simulated dosage) can be processed using the plasma level prediction model to obtain the simulated population data comprising a simulated plasma level metric. FIG. 7 illustrates a sub-sample of simulated population data comprising: a simulated dose 742, simulated patient data 744 (weight, CRCL, ethnicity (Asian vs non-Asian), presence/absence of a P-gp inhibitor, and whether the patient has NVAF) and a simulated plasma level metric 746 such as trough plasma level at steady state.

Following generation of the simulated population data, the ML dosage calculator can be trained using the simulated population data. The ML dosage calculator may comprise any known ML architecture such as an artificial neural network. In some examples, real patient data, e.g. from a clinical study or an ongoing dosage and monitoring program, may be added to the simulated population data to form the ML training data. The real patient data may comprise patient dosage, patient input data and measured drug plasma levels. The ML training data may comprise weightings for each patient data set, with a higher weighting assigned to real patient data than to simulated patient data.

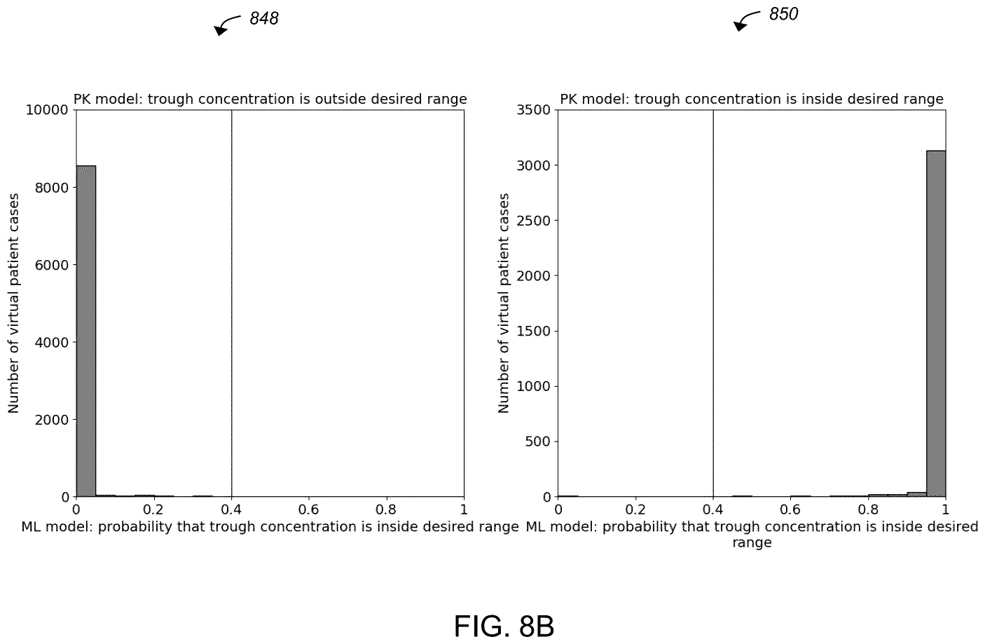

In some examples, the ML dosage calculator may replicate the plasma level prediction model and determine plasma level metrics for a particular dose and then revise the dosage estimate to obtain a dosage for administering to a patient (in the same way as FIG. 5). FIG. 8A illustrates the evaluation results of a dense neural network-based ML regression dosage calculator. The ML dosage calculator was trained on simulated population data for 28,000 simulated patient cases to predict C_{trough} from the corresponding simulated patient data (administered dose and patient characteristics). The simulated population data (training data) was generated using plausible individual demographic, physiological or interacting drug combinations for the simulated patient data, for example: body weight from 42 to 122 kg, CRCL from 45 to 95 ml/min, and with the presence/absence of P-gp inhibitors, Asian ethnicity, and NVAF at frequencies comparable to those in the dataset used to build the Krekels et al. (2016) model. The simulated population data was obtained using the plasma level prediction model described above in relation to equations 1 to 13. The ML dosage calculator was then tested on further simulated population data for 12,000 simulated patient cases. FIG. 8A shows the evaluation of this ML regression model. The figure plots C_{trough} values for the 3,000 simulated patients in the test dataset, as calculated by the plasma level prediction model, against the corresponding C_{trough} values, as calculated by the ML model.

In some examples, the ML dosage calculator may comprise a ML classification model—to predict whether a patient's C_{trough} value falls within a target range (20-30 ng/ml), for a particular dose and their corresponding patient data. FIG. 8B illustrates evaluation results from a ML classification model that was trained and tested on the same data as the ML regression model, except the C_{trough} values in the regression model datasets were categorised into 0 or 1, depending on whether the values fell outside or inside the target range, respectively.

FIG. 8B is divided into two plots: (i) a first plot 848 illustrating the ML classification model's prediction of the probability that each virtual patient's C_{trough} falls within the target range where the plasma level prediction model has calculated that the Ctrough falls outside the target range; and (ii) a second plot 850 illustrating the ML classification model's prediction of the probability that each virtual patient's C_{trough} falls within the target range where the plasma level prediction model has calculated that the C_{trough} falls within the target range. A threshold of 0.4 is set on the x-axis scale to divide the classification into optimal and non-optimal doses—i.e. doses expected to land a virtual patient's C_{trough} within or outside the target range, respectively. This threshold was selected to balance the false negative and false positive rates. In other words, where the ML classification model predicts that the probability that a virtual patient's C_{trough} falls within the target range is less than or equal to 0.4, the final ML prediction is that the virtual patient's C_{trough} falls outside the target range. Similarly, where the ML classification model predicts that the probability that a virtual patient's C_{trough} falls within the target range is greater than 0.4, the final ML prediction is that the virtual patient's C_{trough} falls inside the target range. The corresponding data in Table 2 illustrates that The ML classification model correctly identifies dosages as optimal or not for 99% of the simulated population data. While not infallible, it will be appreciated that further refinements and improvements can be made, such as improvement of training data (more data, more realistic data, real data), and that the performance may be superior to the subjective assessment of dosage by a HCP (e.g. a clinician) who may not have a complete picture of the patient data.

TABLE 2
Classification mapping between PK model (equations 1-13)
and ML model corresponding to data shown in FIG. 8B.

| | |
|---|---|
| ML model: C_{trough} | ML model: C_{trough} |
| outside range | inside range |

| | | | | |
|---|---|---|---|---|
| PK model: C_{trough} | 8,699 | (>99%) | 40 | (<1%) |
| outside range | | | | |
| PK model: C_{trough} | 15 | (<1%) | 3,246 | (>99%) |
| inside range | | | | |

As noted above, a ML dosage calculator that outputs a plasma level metric by processing a particular dose and patient data can be used in the method of FIG. 5 to determine a dosage for administering to a patient.

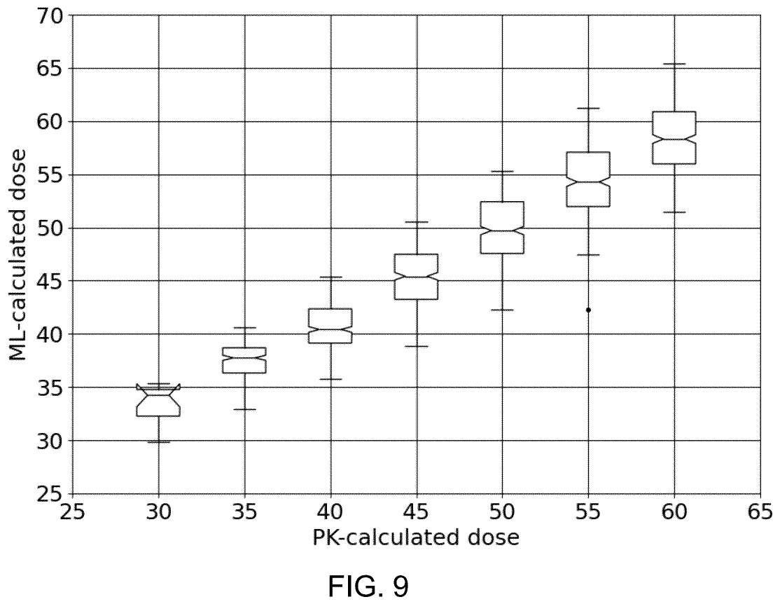

In some examples, the ML dosage calculator may receive a target plasma level metric and patient data, and directly determine a dosage for administering to the patient. FIG. 9 illustrates the validation results of a dense neural network-based ML dosage calculator. The ML dosage calculator was trained on simulated population data for 3,500 simulated patients, each on 7 doses (30, 35, 40, 45, 50, 55, 60, 65, 70 mg). The ML dosage calculator was then tested on simulated population data for 1,500 simulated patients. The simulated population data was obtained using the plasma level prediction model described above in relation to equations 1 to 13. The training and test population data was restricted to data for which the plasma level prediction model indicated a trough plasma level in the range 22.5-27.5 ng/ml. The x-axis corresponds to the dosage used in the plasma level prediction model to provide the trough plasma level in the range 22.5-27.5 ng/ml. The y-axis corresponds to the dosage predicted by the ML dosage calculator to provide the trough plasma level in the range 22.5-27.5 ng/ml. The vertical spread at each point on the plot corresponds to the accuracy of the ML dosage calculator with respect to the plasma level prediction model. In view of the tight range of the target trough plasma level, The ML dosage calculator only outputs a limited spread in recommended dosage indicating strong performance of the direct ML dosage calculator.

A major advantage of the ML dosage calculator is that it can operate at significant speed relative to the two-compartment differential equation model (equations 1 to 13) underpinning the training data. The efficient processing means the ML dosage calculator requires much less processing power than the model of equations 1 to 13 enabling deployment of the ML dosage calculator at scale to HCPs and/or patients, e.g. via a cloud platform and/or personal computing devices. Other advantages of the ML dosage calculator include: (i) a wealth of data available at speed including optimised dosages and plasma level metrics, such as trough levels, Cₘₐₓ, ratios or time profiles (similar to the profiles of FIG. 4A). Such time profiles can be used to indicate a time dependent haemorrhage or clotting risk to a patient (see “Outputs” section below); and (ii) precise, personalised and accurate dosages. Furthermore, the model allows for continuous learning, including incorporation of new adjustment parameters as data accrues. Such adjustment may be performed in regulated phases or stages to ensure ongoing regulatory compliance and safety.

In some examples, a ML dosage calculator may be locked following training and validation, in that the ML dosage calculator will not “learn” from any future data. Such an approach can improve safety and enable easier regulatory approval by ensuring that the ML dosage calculator does not evolve into an unsafe regime based on an error in further training data. Alternatively, in some examples, such as applications where safety requirements may be more relaxed, for example where the drug is used in terminal illnesses, a highly monitored environment, or a clinical situation in which the HCP has no alternative, the ML dosage calculator may be free to evolve and use live data as further training data to provide a more accurate dosage calculator.

The above description relating to the generation, use and advantages of a ML dosage calculator is not limited to edoxaban. Embodiments of the present disclosure can include the above described generation, use and advantages for any drug whose absorption and clearance can be modelled by pharmacokinetic modelling and/or pharmacodynamic modelling such as two-compartment, differential equation-based models. For example, other drugs may be modelled using similar PK-PD models to those of equations 1-4. Specific refinements, similar to equations 5 to 13 can be provided by corresponding real patient studies and PK-PD analysis for the corresponding drug and contributing individual patient data (which may differ from the patient data listed for edoxaban). In some examples, the central compartment may represent locations other than blood plasma, such as the liver, muscle tissue, the brain etc. As a result, the dosage calculator may be generated from a PK-PD model for the drug that predicts a time dependence of an appropriate PK-PD metric. For edoxaban the PK-PD metric is a plasma level metric. However, the PK-PD metric could include other metrics such as drug concentration in the central compartment (e.g. liver, muscle tissue, etc). A ML dosage calculator trained using simulated population data from a two-compartment model advantageously produces an efficient dosage calculator that does not suffer from the huge processing requirements of the underlying differential equation-based model and can therefore be deployed at scale to patients and/or HCPs. In particular, embodiments of the present disclosure include the method of FIG. 6 for any drug that can be simulated using PK and/or PD modelling, including a two-compartment model.

A further advantage of training a ML dosage calculator with simulated population data from a verified PK-PD model relates to the safety, confidence and regulatory approval of ML models. As illustrated by the above examples, a large number of patients may be required to successfully train and maximise the advantages of a ML dosage calculator. Obtaining patient data for the equivalent number of real patients for a particular drug would be infeasible or at best prohibitively time consuming and expensive. As a result, ML models can struggle to obtain regulatory approval, particularly if part of the training phase is proposed to be conducted as part of deployment because such an approach lacks predictability. Embodiments of the present disclosure relate to training and verifying a ML model with a large simulated patient data set that is based on a verified and accepted analytical model developed from real (and attainable) patient data. The resulting ML dosage calculator is safe, predictable and can be locked to avoid any further learning (with a possible exception for defined and regulatory approved update points).

A yet further advantage of training a ML dosage calculator is that it can provide outputs for combinations of covariates that are not well represented in the original patient data set. In this way, the ML dosage calculator can artificially enrich specific cases of interest and improve the weighting and consideration of less common combinations of characteristics thereby reducing bias and inferior predictions.

Furthermore, deploying the safe, predictable, efficient ML dosage algorithm at scale for patients and HCPs can enable the collection of real patient data (including measured PK-PD metrics such as drug plasma levels (see “Ongoing Treatment Management” section below)). Such deployment would be infeasible with a two-compartment differential equation-based PK-PD model due to the processing constraints. The collected real patient data can then be used to evolve the ML dosage calculator and/or the PK-PD model to further improve the accuracy and precision of the dosage calculator.

In some examples, training the ML dosage calculator may be performed in two steps. In a first pre-training step, the ML dosage calculator may be trained using simulated population data, as described above. A second refinement step may comprise training the ML dosage calculator further using refined training data. In some examples, the refined training data may comprise clinical data from real patients comprising patient data, associated drug dosages and resulting drug plasma metrics. In some examples, the refined training data may relate to a different drug that has a similar PK-PD pathway to the drug used in the first pre-training step. In this way, ML dosage calculators can be pre-trained in a generic way and then refined using real world patient data and/or for a related drug with a similar underlying structure.

### Patient Data

Current regulatory guidelines for edoxaban dosing only account for a limited number of patient parameters, including renal function, body weight, and coadministered drugs. However, the guidelines do not take into account patient demographics such as ethnicity nor the potential for inter-subject variability in drug absorption described above.

The above described plasma level prediction models indicate that a kidney function metric, specifically creatinine clearance, is the largest contributing factor to drug plasma level variability among the patient population. Age, weight, ethnicity, the presence of other medications (specifically, P-gp inhibitors) can also all modulate the circulating drug level in the blood plasma.

Returning to the first step 212 of FIG. 2, in the simplest embodiments, the patient data comprises the kidney function metric. In such embodiments, average population values, or surrogate substituted values, may be used for the other parameters involved in the above dosage calculators and plasma level prediction models. In some embodiments, the patient data may include one or more additional patient parameters, such as weight, ethnicity, the presence of NVAF and other medications (as used in the Krekels replica model described above) or age, sex, other disease status and other potential covariates used in other plasma level prediction models. In some examples, the patient data may include the most easily accessible data, such as age, weight, sex, ethnicity, and co-medications. In some examples, parameters requiring specific medical tests such as creatinine clearance or coagulation tests may be estimated using surrogate substitution as described below.

Patient weight can present a particular issue with present guidelines as international guidelines for edoxaban only specify dosing above and below 60 kilograms with no further specific adjustment for those who are obese, for example a patient with a body weight of 120 kg and a height of 1.93 m. There is also limited evidence for patient body mass index (BMI) exceeding 40. Increasingly, patients weigh more than this limit and there is no knowledge as to what to do for them in terms of DOACs, with many then receiving warfarin with consequent higher risk. By including weight as a parameter of the patient data, the dosage calculator can advantageously account for patients with excessive weight.

Additionally, endothelial changes, with disease or just age, can affect absorption of edoxaban. By including age as a parameter of the patient data, the dosage calculator can advantageously account for the age dependency on plasma drug levels.

The above described plasma level prediction models and calculators can require values for age, sex, creatinine clearance, weight, ethnicity, co-medications, and disease status. While the use of specific interfering drugs is likely to be known, along with weight, age, sex, and disease status, in some circumstances, creatinine clearance may not be known without prior testing.

In some examples, the values of CrCL may be provided by population-based surrogate estimates. For example, an estimated creatinine clearance rate (eCCR) can be provided by the Cockcroft-Gault formula. Creatinine clearance can be estimated using serum creatinine levels. The Cockcroft-Gault (C-G) formula uses a patient's weight (kg) and sex to predict CrCL (mg/dL). The resulting CrCL is multiplied by 0.85 if the patient is female to correct for lower CrCL in females. The C-G formula is dependent on age as its main predictor for CrCL. The C-G formula can be written as:

$$e C C r = \frac{( ( 14 \; 0 - Age ) \times Mass \times [ 0.85 if female ] )}{[ Serum Creatinine ( \frac{mg}{d l} ) ]}$$

Serum creatinine can be measured, derived from age related change and potentially further revised according to other diseases or conditions. In the absence of a direct measurement of CrCL, the eCCR can be substituted for CrCL in the dosage calculations described herein.

### Other Patient Data Parameters

The present disclosure may encompass other plasma level prediction models and dosage calculators that are available now or may be available in the future following appropriate patient studies.

Therefore, according to embodiments of the present disclosure, the patient data may include other patient parameters that may be endothelial function drivers. These include one or more of: smoking history; alcohol intake; a patient thrombosis history; a patient haemorrhage history; a patient cancer history; a patient cardiovascular history; a patient metabolic history; a patient platelet count; patient genetic determinants; a patient haematocrit; a patient liver function; a patient blood pressure; patient co-conditions; patient activity level; patient dosage compliance; family history of thrombosis; family history of strokes; family history of bleeding (e.g. anaemia); and family history of hypertension.

The patient data may also include medications other than those specifically mentioned above, including proton pump inhibitors such as esomeprazole, P-gp inhibitors, calcium channel blockers, verapamil, antifungals such as ketoconazole, antiarrhythmics such as amiodarone or dronedarone, antibiotics such as rifampicin, or drugs which enhance the anticlotting activity such as a nonsteroidal anti-inflammatory drugs, aspirin or clopidogrel.

The patient data may also comprise a patient genomic type. The patient genomic type may comprise a patient genotype for Pgp transporter genes such as ABCG or metabolic enzymes such as cytochrome P450 data. Cytochrome P450 is a suite of drug-metabolizing enzymes which are mainly found in the liver, but can also be found throughout the body. In some examples, an administered drug can reduce or increase the activity of these enzymes which may result in other co-administered drugs having higher or lower plasma levels. In this way, other drugs can have an effect on edoxaban blood levels by modifying how it is metabolised. Therefore, models and calculators utilising cytochrome P450 data can account for drug interactions.

The patient haemorrhage history may include haemorrhage events indicating a severity and time of event. The patient haemorrhage history may relate to the endothelial state or bleeding propensity such as propensity to superficial bruises on arms and legs and other measurements of skin elasticity. Other methods for assessing blood vessel data and/or endothelial state include image analysis of retinal vessels from fundal photography. The flexibility of the model facilitates incorporation of surrogate measures of endothelial state that may be incorporated into future plasma level prediction models, for example by assessment and training using Machine Learning capabilities.

The patient thrombosis history may include thrombosis events indicating a severity and time of event. The family history of thrombosis may include first degree relatives and may include those with an age less than 50 years.

The patient history of at risk events may include recordings of intermittent atrial fibrillation events, including their duration, time of occurrence and rate. Such recordings may be provided from cardiac data from a smart watch.

In relation to cancer history, cancer has a particularly high thrombotic risk because the endothelium becomes very activated, which then releases various coagulation factors. Even with treatment with DOACs, thrombosis can commonly occur in cancer settings, with a high recurrence rate of approximately 17 to 18%, yet also a risk of major haemorrhage of 20%. This is particularly so with certain cancers such as lung, pancreas and colon, demanding even more patient monitoring and precise dosing. Overall, it is estimated that even in the best centres, only one third of patients with thrombosis get well managed and that elsewhere where management is by a general physician or respiratory physician, optimal management is received in less than 10% of cases. As described below, embodiments of the disclosure can include a personalised target plasma level metric for cancer patients to account for their unique PK-PD. As also described below, embodiments of the disclosure may include enhanced monitoring to calibrate and/or personalise the plasma level prediction model and dosage calculator.

Patient genetic determinants may supplant, refine, or improve the model or calculator classification of ethnicity (for example).

The patient activity level may represent personal risk exposure, for example an indication that the patient partakes in hazardous activities or other lifestyle factors, such as recreational drugs, physical activity, etc.

The patient co-conditions may include an indication of whether the patient suffers from AF and an extent of the AF. If the extent of the AF is particularly bad with dizzy spells fainting and tachycardia then higher doses would be warranted.

If the patient has a family history of strokes or hypertension then higher dosing with some bleeding may be more tolerated.

Patient dosage compliance may indicate a patient's propensity to take their medication in accordance with a prescribed regimen. The plasma level prediction model and dosage calculator may be refined to account for patient compliance (e.g. via a future patient trial). The method may include monitoring the patient compliance and adjusting the dosage accordingly.

The patient metabolic history may be indicative of diabetes which can modify coagulation and blood circulation as a comorbidity.

One or more of the above patient parameters may be incorporated into the plasma level prediction model and/or dosage calculator via an appropriate patient trial. Patient data, including the one or more (new) patient parameters, together with patient dosage (amount and time) and directly measured plasma level metrics can be recorded to evolve the plasma level prediction model and/or dosage calculator to account for the additional dependency of the one or more new patient parameters accordingly.

The patient data may comprise yet further patient data parameters. For example patient data parameters may include those that can influence personalised target plasma level metrics as discussed in the relevant section below. Further patient data parameters may include side effect reporting (see section “Side Effect Monitoring” below), a time a dose was taken and other examples described herein.

### Receiving Patient Data

Patient data may be received by one or more of: manual data entry, by either a patient, HCP or third party at a computing device such as a personal computing device; data from medical records stored on a database or similar; and receiving physiological measurements, for example from medical devices or clinical databases. For example, patient data may include cardiac data from a smartwatch that can indicate periods of AF.

### Implementations

The method of FIG. 2 may be implemented as one or both of: (i) a software based dosage calculator configured to determine a relational mapping between starting dosage, patient data and target plasma level metric; and (ii) a multi-use deployed software based dosage calculator configured to indicate dosages in a short timeframe (sub-seconds, seconds or minutes) to a health practitioner and/or a patient via a personal computing device or the like.

### Relational Mapping Implementation

A relational mapping between patient data, dosage and one or more plasma level metrics may take the form of a look-up table, a nomogram, an analogue computer or the like. Any of the above described dosage calculator examples (or a combination of them) may be used to determine the relational mapping. For example, a detailed relational mapping can be determined by performing the methods of FIG. 5 or FIG. 6 for a population set of different patient data. The population set of patient data may include simulated patient data and/or real patient data. The resulting output may include a detailed mapping between one or more target plasma level metrics (e.g. ITL) and starting dose for a range of the patient data variables (e.g. kidney function, age, ethnicity, sex, medications etc). The relational mapping (e.g. look-up tables) can provide one or more of: (i) a dosage calculator for the implementation of the method of FIG. 2 (i.e. quick dosage calculation for patient/HCP); (ii) a training data set for a ML dosage calculator; (iii) the foundations of a clinical guidelines reference; and (iv) standard dosing regimens for edoxaban products, labelling, packaging etc.

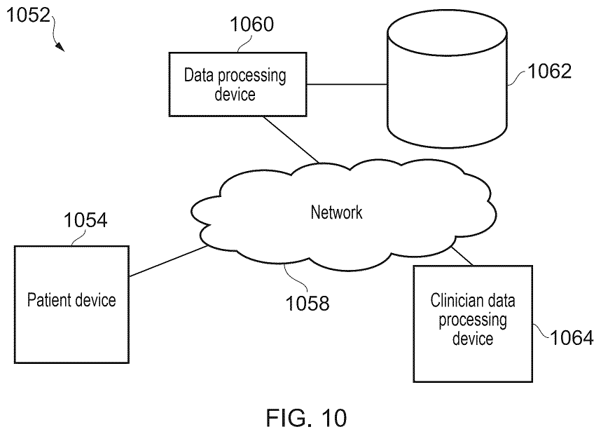

The relational mapping implementation may be implemented using any known computing device architecture such as a stand-alone computing system or a networked computing system such as that of FIG. 10 described below in relation to the second implementation.

### Multi-Use Deployed Software Implementation

FIG. 10 illustrates a system suitable for carrying out the multi-use deployed software implementation (also referred to herein as the digital app) and any of the computer implemented methods described herein, including the method of FIG. 2. System 1052 includes a patient device 1054. The patient device 1054 may comprise one or more processors configured to: receive the patient data; process the patient data with a dosage calculator to determine the dosage of edoxaban for administering to the patient, wherein the dosage calculator is derived from a plasma level prediction model; and indicate the dosage.

In the illustrated embodiment, patient device 1054 is a smartphone. However, the invention is not limited in this respect and the patient device 1054 can take many other forms, including but not limited to a mobile telephone, a tablet computer, a desktop computer, a voice-activated computing system, a laptop, a gaming system, a vehicular computing system, a wearable device, a smart watch, a smart television, an internet of things device and a medicament-dispensing device.

The patient device 1054 may be configured to gather one or more parameters of the patient data. The patient data can be obtained via manual data entry using a human interface device of patient device 1054 and/or from a remote source via the network 1058.

The patient device 1054 may comprise a memory (not illustrated) for storing the patient data and/or outputs of the dosage calculator. Such data may also be stored on a database 1062 as networked or cloud-based data storage.

The patient device 1054 may have one or more applications (or apps) installed on a storage medium associated with the patient device (not shown). The one or more apps may be configured to perform any of the computer implemented methods disclosed herein. The one or more applications may be configured to assist the patient in providing the patient data and/or may include the dosage calculator for processing the patient data. The one or more applications may be downloaded from a network, for example from a website or an online application store.

In this example, the system 1054 further comprises a data processing device 1060 that is communicatively coupled to the patient device 1054 via the network 1058. In the illustrated embodiment network 1058 is the internet, but the invention is not limited in this respect and network 1058 could be any network that enables communication between patient device 1054 and data processing device 1060, such as a cellular network or a combination of the internet and a cellular network.

The data processing device 1060 may supplement the patient device 1054 and perform one or more steps of any of the computer implemented methods disclosed herein. For example, in some embodiments, the patient device 1054 may receive the patient data and provide the patient data to the data processing device 1060. The data processing device 1060 may then process the patient data with the dosage calculator to determine the dosage for administering to the patient. The data processing device 1060 may then provide the dosage to the patient device 1054 or a HCP device 1064 (also referred to as clinician device 1064) either or both of which may indicate the dosage. In this way, the data processing device 1060 provides networked, server based or cloud based, processing capability to the system for performing the computer implemented methods.

The data processing device 1060 may be coupled to the database 1062 that can store the patient data and/or the outputs of the dosage calculator.

In this example, the system 1052 includes a clinician data processing device 1064 that is communicatively coupled via network 1058 to the patient device 1054 and the data processing device 1060. The clinician data processing device 1064 may be broadly similar to patient device 1054, offering a similar set of functionality. Specifically, the clinician data processing device 1064 enables patient data to be collated or received. Clinician data processing device 1064 is contemplated as being physically located at a HCP's premises during its use, such as a clinic, a doctor's surgery, a pharmacy or any other healthcare institution, e.g. a hospital. Clinician data processing device 1064 may include one or more sensors, and/or be configured to control one or more separate sensors, which sensors are capable of gathering information about the patient, e.g. a blood pressure sensor.

It is also contemplated that clinician data processing device 1064 is typically used by a medically trained person with appropriate data security clearance, such that more advanced functionality may be available than via the patient device 1054. For example, the clinician data processing device 1064 may be able to access a medical history of the patient, generate an edoxaban prescription for the patient, place an order for medication, etc. Access to functionality may be controlled by a security policy implemented by a local processor or data processing device 1060.

The data processing device 1060 and/or the clinician device 1064 may have an application installed that is compatible with or the same as the application installed on the patient device 1054.

It will be appreciated that the various steps of the computer implemented methods disclosed herein may be performed in any combination by any of the one or more processors in the patient device 1054, the data processing device 1060 and the clinician device 1064. For example, all steps may be performed by the clinician device 1064 which receives one or more parameters of the patient data locally, from the patient device 1054 or another remote device via the network 1058 and optionally via the data processing device 1060. In a further example, all steps may be performed in a networked back-end on data processing device 1060, with patient device 1054 and clinician device 1064 acting as human interfaces for gathering and indicating data. In a yet further example, all steps may be performed on patient device 1054 with clinician device 1064 merely gathering relevant data from the patient device 1054 for informing or directing the HCP.

It will also be appreciated that one or more of the components of the system 1052 could be omitted depending upon the application. For example, at a clinic setting, the disclosed computer implemented methods could be performed solely on the clinician device 1064. Alternatively, the methods could be performed solely on the patient device 1054 in a domestic setting.

The dosage calculator may be deployed to any of patient device 1054, data processing device 1060, and clinician device 1064 as a digital app. The digital app may indicate to the HCP a dose (starting dose, continuing dose or revised dose, timing of dose) for administering to a particular patient based on computed changes in drug levels to achieve the Ideal Therapeutic Level (ITL).

The digital app may also summarise important factors (other patient data) that affect thrombosis and bleeding and present this to the HCP. These factors can include thrombosis and haemorrhage history, cancer history, pattern of any falls, pattern of renal function and liver function, pattern of platelet count, haematocrit, platelet and blood transfusions and age of patient. The app may also recommend a follow up pattern and prompt for any missing information.

### Outputs

### Dosage Outputs

In relation to the second and third steps of FIG. 2 relating to determining and indicating the dosage for administering to the patient, the dosage determination and indication may be performed in a number of ways.

The indicated dosage may comprise a starting dosage for a patient beginning edoxaban treatment, a continuing dosage confirming that a patient taking edoxaban is dosed at a correct level, or a revised dosage suggesting that a patient's dosage should be adjusted in view of updated patient data (discussed further below under “Ongoing Treatment Management” section).

Wherever dosage is used, this refers to both amount administered, form of medicament (e.g. immediate or controlled release), time taken in relation to food and other events, and periodicity.

In some examples, determining the dosage may comprise: (i) processing the patient data with the dosage calculator to determine an ideal dosage regimen; and (ii) selecting the dosage for administering to the patient from a selection of available dosage regimes, based on the ideal dosage regimen. For example, the second step of the method of FIG. 2 may comprise selecting a dosage amount from the available dosage amounts, such as 15, 30, and 60 mg. For some patients, the method may determine and indicate a higher dosage at night and a lower dosage in the morning, so that Cₘₐₓ occurs at a lower risk time (e.g. in a patient with frequent falls), with the method computing the optimal overall dosage to achieve sufficient clot prevention at all times.

As described below, the present disclosure encompasses novel dosage forms that increase the flexibility in selecting the dosage amount and selecting the dosage amount may comprise selecting dosage amounts that are: increments of 0.5 mg, 1.0 mg, 5.0 mg, 10 mg or 25 mg up to a maximum dosage amount of 120 mg. For example, the dosage amount may comprise any of: 5, 10, 20, 25, 50, 75, 90, or 120 mg in addition to those already available (15, 30 and 60 mg). Selecting the dosage regimen may also comprise selecting dosage forms such as liquid formulations or microgranule formulations as described below.

Determining and indicating the dosage regimen may comprise determining and indicating one or more of: a dosage amount; a dosage time; a dosage frequency; and/or a dosage type. The dosage frequency may comprise once daily as is the current standard for edoxaban. However, the disclosure also encompasses other dosage frequencies including less than daily or multiple times daily which can reduce the maximum plasma level, Cₘₐₓ. The method can determine and indicate that for certain patients the medication should only be taken every other day (for example if creatinine clearance is very low), or twice per day (for example if creatinine clearance is very high). Or for other patients the method can determine and indicate that daily dosing will result in unacceptably risky Cₘₐₓ values to achieve a desired C_{trough}, and therefore either a slow-release formulation should be given or an increased frequency of dosing with a lower dosage form.

The dosage time may comprise a time for the patient to take their medication. The dosage time may be provided as simple reminder prompts to a patient to take their medication. The dosage time may also be specific to an event such as upcoming or recently performed surgery. The dosage type may relate to different dosage forms such as solid dose, liquid dose or microgranule formulation.

### Novel Dosage Forms

The disclosed plasma level prediction models, dosage calculators, digital apps and associated methods can encompass dosage amounts, formulations and regimens beyond those currently approved.

As noted above, there are only three doses of immediate release forms of edoxaban available 15, 30 and 60 mg.

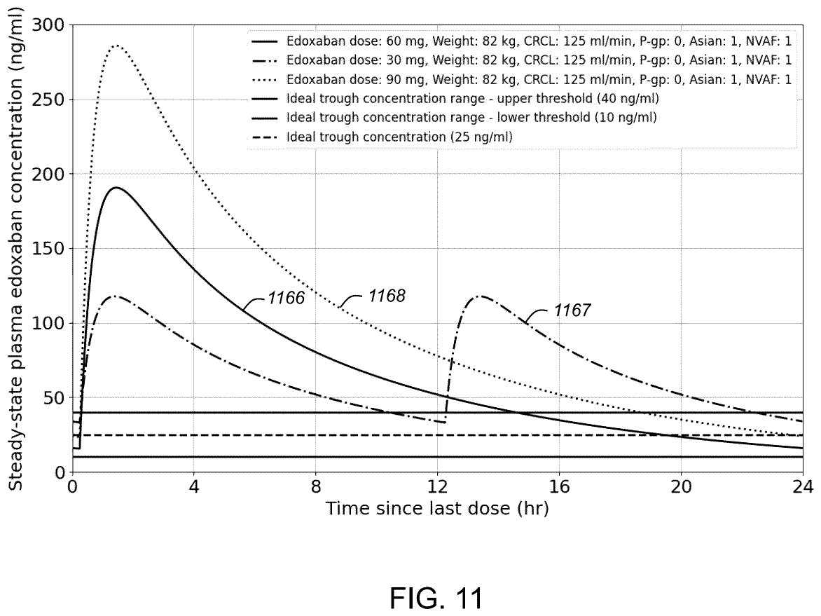

Thus the need for other doses is apparent such as 5, 10, 20, 25, 50, 75, 90, or 120 mg in addition to those already available which can provide the right dose for the right patient using the app algorithms described above. FIG. 11 illustrates the estimated plasma level time profile 1166 at day 10 for a simulated patient (82 kg, CRCL 125 ml/min, no P-gp inhibitor, Asian, with NVAF) taking a dosage of 60 mg of edoxaban once daily. As can be seen, the simulated patient's C_{trough} falls towards the lower end of the ITL of 10-40 ng/ml on 60 mg. FIG. 11 also illustrates the estimated plasma time profile 1167 at day 10 for the same patient on 30 mg edoxaban taken twice daily. As can be seen, changing the dose to 30 mg twice daily causes the simulated patient's C_{trough} to fall towards the upper end of the ITL. Although 30 mg is available for the treatment of DVT and PE, the US prescribing information for SAVAYSA (branded edoxaban) [3] does not explicitly recommend reducing the dose to 30 mg in patients with NVAF. Therefore, in this sense, 30 mg is a non-available dose. FIG. 11 also illustrates the estimated plasma time profile 1168 at day 10 for the same patient on 90 mg edoxaban taken once daily. As can be seen, changing the dose to 90 mg once daily causes the simulated patient's C_{trough} to land on the ideal plasma concentration of 25 ng/ml.

The figure illustrates how the disclosed systems and methods may enable administration of edoxaban for patients where prescribing guidelines currently recommend that edoxaban should not be prescribed. For example, the US prescribing guidelines suggest edoxaban should not be used in patients with a creatinine clearance >95 ml/min because of increased risk of ischaemic stroke compared to warfarin (at the highest dose studied (60 mg)). For the 90 mg and 2×30 mg dose curves 1167, 1168, the patient spends less time at low plasma levels of edoxaban, potentially lowering their risk of thrombosis/ischaemic stroke. However, a phase II trial (NCT00504556, Weitz et al. 2010 [9]) found that the risk of bleeding was greater in patients with NVAF taking 30 mg twice-daily vs 60 mg once-daily for 12 weeks, potentially due to the elevated C_{trough} (despite the reduced Cₘₐₓ). However, the 30 mg twice-daily regimen may be beneficial for a patient at high risk of thrombosis/ischaemic stroke but at a low risk of bleeding (e.g. using the HAS-BLED score). Further clinical data may elucidate this.

The figure illustrates one example where doses other than those presently available may be most suitable for a given patient. Other examples include where lower doses may be more suitable before and after surgery or where a patient exhibits all the indices for prolonged retention of the drug in the body, or to reflect the changing risk of thrombosis in an individual with time.

The disclosed plasma level prediction models, dosage calculators, digital apps and associated methods can account for other dosage amounts as described above. Indeed, the disclosed systems and methods can perform better when more precise dosage amounts are available.

The disclosed methods and calculators may also determine a dosage regimen that increases the frequency of dosing to achieve optimal trough levels without exceeding a threshold value of Cₘₐₓ. The inventors have realised such a capability is desirable because haemorrhage risk is more related to Cₘₐₓ in contrast to thrombosis risk which is more related to overall drug exposure and hence C_{average}, AUC or blood trough levels. Overall it may be desirable to minimise the Cₘₐₓ/C_{trough} ratio, and avoid Cₘₐₓ exceeding certain levels. Computing such profiles for particular patients has hitherto not been practically possible, but is enabled by the methods and apparatus disclosed herein.

All of the available doses are immediate-release producing peak levels after approximately 1-2 hr when taken without food and an hour later with food. Since the terminal elimination half-life following oral administration is only ˜10-14 hr, marked change from Cₘₐₓ to trough levels are seen with the currently recommended once-daily dosing. A second dose could be given to achieve more stable plasma levels, but multiple dosing can result in reduced patient compliance. Therefore, there is a need for the development of sustained release formulations. Sustained release formulations would have the advantage of not only allowing once-daily dosing but also reducing the maximum drug plasma level, Cₘₐₓ, which is associated with bleeding episodes.

The terminal elimination half-life of edoxaban following oral administration is approximately 10-14 hr and although once-daily dosing is recommended, this will lead to a high peak-to-trough ratio. Controlled release formulations may therefore be developed for such to reduce the unnecessary large peaks and thus reduce the potential for bleeds, for example using a microgranule formulation (or any other known formulation for controlling solubility and half-life) which can be taken once a day to flatten out the sharp peaks of plasma drug levels. Each microgranule may exhibit a controlled release profile and development of multiple doses may be facilitated by altering the number of microgranules in the capsule without having to reformulate and test for each individual dose.

The disclosed models, calculators, apps and methods can accommodate such changes to dosage form, potentially following clinical trials to refine and validate the plasma level prediction model and/or dosage calculator.

### Plasma Level Metric Outputs

Embodiments of the dosage calculation method, particularly those of the digital app implementation, may also process the patient data to determine and optionally output one or more plasma level metrics. The dosage calculation method may comprise determining one or more of: a maximum plasma level (Cₘₐₓ); a trough blood plasma level; a ratio of the maximum blood plasma level to the trough blood plasma level; a plasma level time profile; an area under the curve of the blood plasma level profile; a ratio of the maximum blood plasma level to the area under the curve of the blood plasma level profile; and the average daily plasma level at steady state, C_{average}. The dosage calculation method may comprise outputting one or more of these plasma level metrics to the patient or HCP.

As discussed further below (under the “Personalised Target Plasma Level Metrics” section), the maximum plasma level, the trough plasma level and/or the ratio between these may be used to adjust the target plasma level metric to ensure an optimal balance between bleeding risk and thrombosis risk. Outputting these plasma level metrics to a HCP can enable the HCP to manually adjust the target plasma level metric.

In some embodiments, the dosage calculation method may comprise outputting the plasma level time profile or risk levels derived from the plasma level time profile. The plasma level time profile may comprise data similar to that represented in FIG. 4A representing the variation of drug plasma level with time. In such examples, the patient data may include a dosage time at which the patient received a dose of edoxaban. The dosage time can then be used as a start time for the plasma level time profile.

The digital app may use the plasma level time profile to determine and indicate a patient thrombosis risk and/or a patient haemorrhage/bleeding risk according to the plasma time level profile and the dosage time. The digital app may translate circulating drug plasma level to risk based on predetermined plasma level-risk relationships such as those illustrated in FIGS. 1B and 1C.

Indicating the bleeding risk may comprise indicating to the patient (e.g. via the digital app), times of day at which they are at increased or reduced risk of haemorrhage and thus able to adapt their behaviours accordingly.

In some examples, a HCP portion of the app may output the bleeding risk, the plasma level time profile and/or a circulating drug plasma level (derived from the time profile) to a HCP in advance of an invasive procedure. Such information provided to the HCP can guide timing of procedures and how long a medication should be withheld before a particular invasive procedure, such as surgery, and how soon a procedure can be carried out. In some examples, the digital app may indicate a time that the patient or HCP should wait before undergoing an invasive procedure (or changing medication to a different anticoagulant). The output plasma level metrics can be used to determine if the edoxaban plasma levels are suitable for surgery, for example where a cut-off of ˜30 ng/ml is thought to be acceptable, aiding the clinical decision. It is recognised that different surgeries carry different risks and the thresholds may be adapted accordingly.

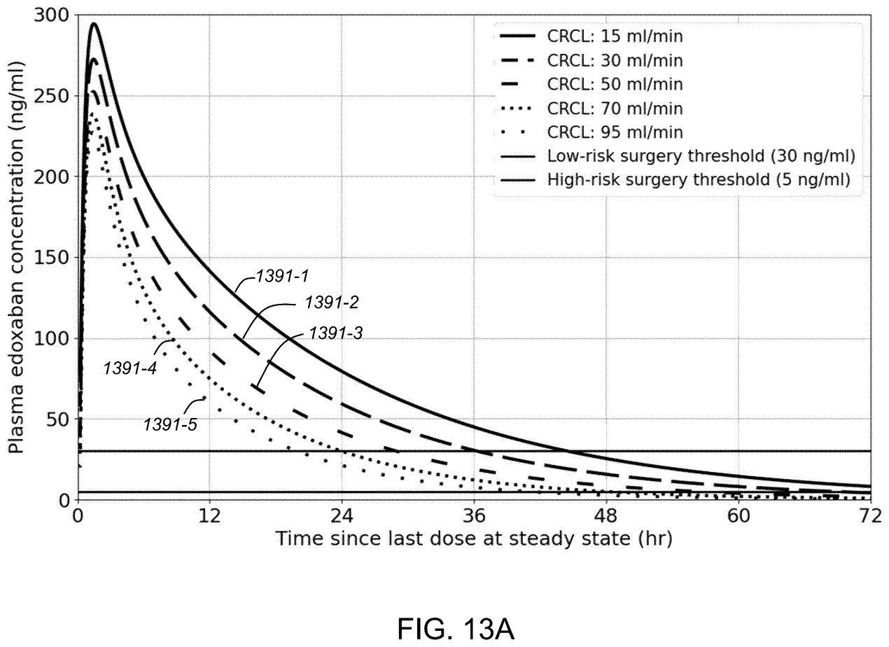

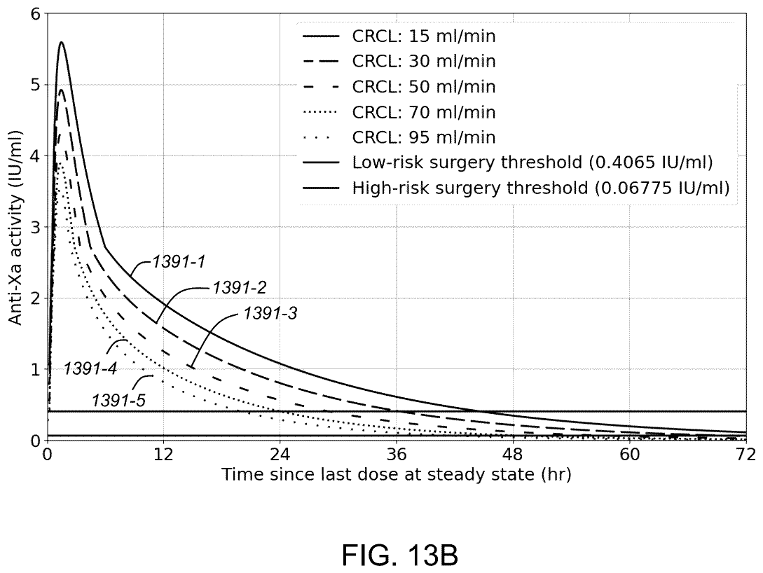

FIG. 13A schematically illustrates the determination of procedure wait times according to an embodiment of the present disclosure. The procedure wait time can define a time from the time of the final dosage of edoxaban prior to an invasive procedure until the plasma level has fallen below the invasive procedure plasma level threshold. The invasive procedure plasma level threshold comprises a plasma level deemed safe for the invasive procedure (i.e. edoxaban levels have sufficiently reduced to avoid major haemorrhaging).

The figure illustrates time-dependent plasma level profiles calculated using the second example dosage calculator (i.e. directly using the plasma level prediction model) for patients following a final 60 mg dose of edoxaban. The figure illustrates five time-dependent plasma level profiles: a first plasma level profile 1391-1 for a first subject with severely impaired kidney function CrCL=15 ml/min; a second plasma level profile 1391-2 for a second subject with significantly impaired kidney function CrCL=30 ml/min; a third plasma level profile 1391-3 for a third subject with moderately impaired kidney function CrCL=50 ml/min; a fourth plasma level profile 1391-4 for a fourth subject with slightly impaired kidney function CrCL=70 ml/min; and a fifth plasma level profile 1391-5 for a fifth subject with healthy kidney function CrCL=95 mL/min. The figure illustrates high-risk and low-risk invasive procedure plasma level thresholds of 5 ng/ml and 30 ng/ml respectively. The procedure wait time for each patient for a high-risk/low-risk procedure may correspond to the time the respective plot falls below the respective invasive procedure plasma level threshold (30 ng/ml or 5 ng/ml).

In some examples, the patient data includes a final drug dosage prior to drug withdrawal in advance of an invasive procedure and the app/method processes the final drug dosage with the dosage calculator to determine a procedure wait time for the plasma level to fall below an invasive procedure plasma level threshold. The final drug dosage may include a dosage amount and/or a dosage time. In some examples, this procedure wait time calculation may be performed independently of any dosage calculation and may be performed for any DOAC.

In some examples, the app/method may estimate a prothrombin time based on the plasma level concentration and determine the procedure wait time for the prothrombin time to fall below an invasive procedure prothrombin time threshold. In some examples, the invasive procedure prothrombin time threshold comprises 13 seconds. In some examples, the app/method may determine the procedure wait time for the prothrombin time to fall within an invasive procedure prothrombin time threshold range, which may be 10 to 13 seconds. The app may estimate the prothrombin time from a calculated plasma level concentration using known relationships between the two quantities, as described above and below in relation to FIG. 1A and ongoing treatment management.

In some examples, the app/method may estimate an anti-Xa activity based on the plasma level concentration and determine the procedure wait time for the anti-Xa activity to fall below an invasive procedure anti-Xa activity threshold. The app may estimate the anti-Xa activity from a calculated plasma level concentration using known relationships between the two quantities, for example as described above in relation to FIG. 1A and below in relation to equations 14 and 15 defined in the section ‘ongoing treatment management.’ In some examples, the invasive procedure anti-Xa activity threshold may comprise a low-risk invasive procedure anti-Xa activity threshold. The low-risk invasive procedure anti-Xa activity threshold may comprise 0.4065 IU/ml corresponding to a plasma edoxaban concentration of 30 ng/ml (according to equations 14 and 15). In some examples, the anti-Xa activity threshold may comprise a high-risk invasive procedure anti-Xa activity threshold. The high-risk invasive procedure anti-Xa activity threshold may comprise 0.06775 IU/ml corresponding to a plasma edoxaban concentration of 5 ng/ml. The thresholds are exemplary and may change depending on access to new data, different indications, different assay kits with different reagents, etc. FIG. 13B illustrates the variation in anti-Xa activity 1393-1, 1393-2, 1393-3, 1393-4, 1393-5, in the hours following the final 60 mg dose of edoxaban received by the first to fifth patients of FIG. 13A. The invasive procedure low-risk and high-risk anti-Xa activity thresholds are also highlighted.

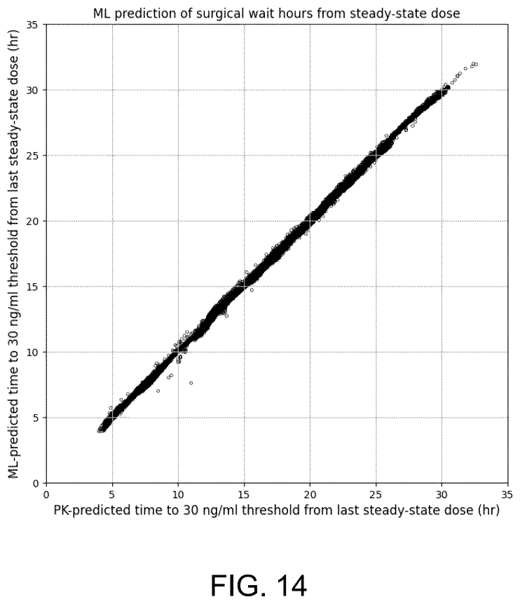

In some examples, a ML dosage calculator may be used to determine the procedure wait time. The ML dosage calculator may be trained using simulated population data comprising simulated patient data (as described above) and simulated procedure wait times calculated with the plasma level prediction model/second dosage calculator. The ML dosage calculator may be trained for a specific invasive procedure plasma level threshold or may be trained for a range of invasive procedure plasma level thresholds. The trained ML dosage calculator may: receive patient data including a final drug dosage; and process the patient data to determine a procedure wait time for the plasma level to fall below an invasive procedure plasma level threshold. FIG. 14 plots the predicted procedure wait time calculated with a ML model versus the predicted procedure wait time calculated with the plasma level prediction model. The figure illustrates excellent agreement between the two approaches. A ML based dosage calculator for predicting surgical wait times realises the same advantages as the ML based dosage calculator described above, namely the ability to deploy the calculator at scale for rapid calculations because there is no requirement to process the differential equations of the plasma level prediction model.

Table 3 illustrates plasma concentrations for patients with different levels of CrCl, if the wait time after the last dose prior to surgery was the minimum recommended by EHRA, for both low and high bleed-risk procedures. The table illustrates the wide range of plasma concentrations present ahead of high-risk surgery depending on kidney function. The disclosed methods and systems can advantageously account for this variation by providing personalised procedure wait times, as described above.

TABLE 3
Plasma concentrations after EHRA recommended
wait time for invasive procedure

| | Plasma concentration after | Plasma concentration after | |
|---|---|---|---|
| | EHRA-recommended pre- | EHRA-recommended pre- | |
| CrCL | surgery (low risk) minimum | surgery (high risk) minimum | |
| (mL/min) | waiting time (ng/ml) | waiting time (ng/ml) | |
| 95 | 21.1 (after 24 hr) | 2.7 | (after 48 hr) |
| 70 | 29.9 (after 24 hr) | 4.9 | (after 48 hr) |
| 50 | 41.4 (after 24 hr) | 8.6 | (after 48 hr) |
| 30 | 59.1 (after 24 hr) | 15.6 | (after 48 hr) |
| 15 | 44.8 (after 36 hr) | 25.3 | (after 48 hr) |

In some examples, a patient facing portion of the digital app may indicate the thrombosis risk including advising isolated periods of thromboprophylaxis for events where they are at higher risk of a further thrombosis, for example, surgery or flights of longer than four hours duration, after the patient has completed regular anticoagulation for the original clot. The app could advise on when to increase back to therapeutic anticoagulation from prophylactic after pausing anticoagulation for a planned procedure. This latter aspect could be either presented in a patient facing app, and/or a clinician facing app.

In some examples, the digital app may receive patient data from other objective monitoring systems. The digital app may receive activity data, e.g. from wearable devices, to monitor activity levels and impute associated risk elevation or reduction. Increased activity confers lower risk of stroke, but must not exceed a level that bleeding risk is unacceptably elevated. The digital app may also receive blood pressure data from a blood pressure monitoring device. Blood pressure is linked with the probability of stroke, including haemorrhagic stroke. The digital app may adjust or scale the bleeding risk or thrombosis risk based on the blood pressure data and/or the activity data. Incorporating home blood pressure monitoring data into the patient data and providing it to the HCP may also be useful to help the HCP decide if additional treatment is necessary, something that is not routinely undertaken in patients receiving DOACs.

### Personalised Target Plasma Level Metrics

The tendency to clot can be influenced by a variety of patient parameters that vary with time and between individuals, such as those already described above and in addition, hydration state, infection, inflammation, menstruation, blood pressure and the rate of blood flow through blood vessels. Sluggish blood flow can be seen with prolonged immobility, and in particular after surgery. Increased risk of haemorrhage can occur if there is trauma or a medical procedure involving penetration of a needle, or surgery. As already described, blood vessel walls can become weaker as a result of ageing and in particular deposition of amyloid as occurs in the brain, or from inflammatory processes or drugs such as steroids or antidepressants. The beginnings of clot formation may occur as part of the normal range of physiology, but anticoagulant properties of the coagulation cascade prevent extension.

The inventors have realised that the process of clot formation, or thrombosis, takes place over a longer time course than the process of haemorrhage. The balance between thrombosis tendency and haemorrhage tendency will vary between individuals based on their particular body state. The tendency can be shifted by dosing of DOACs. The longer process of thrombus formation is more closely related to the total exposure to the DOAC, whereas the haemorrhage tendency is more related to the maximal drug concentration. Therefore, consideration of both of these parameters and the ratio thereof offers a route to a more personalised approach to anticoagulation to achieve the best risk-benefit profile. For example, one approach may be to minimise the ratio of the maximum plasma level, Cₘₐₓ, to the area under the plasma level time profile. As noted above, current formulations can only suppress Cₘₐₓ by prescribing multiple doses per day which brings its own problems of compliance. The solution of a controlled release product that can be used once daily, while desirable, is not yet available.

Disrupting the above balance of risk, and exacerbating incorrect dosing of anticoagulants, is an asymmetry in prescribing tendency amongst HCPs reflecting the psychological desire for blame avoidance. HCPs may perceive the occurrence of a thrombosis which then may lead to an embolism as less blameworthy than occurrence of a haemorrhage (omission vs commission). As a consequence, HCPs may tend towards underdosing to avoid the risk of a potential overdose, even though at a population level this leads to worse outcomes. This reflects a psychological flaw rather than rational prescribing.

Embodiments of the dosage calculation method, particularly those of the digital app implementation, may account for individual clotting and bleeding risk factors by determining the target plasma level metric as a personalised plasma level metric. In some examples, the target plasma level metric may comprise a trough plasma corresponding to the ITL. The personalised plasma level metric may comprise an adjustment to the ITL. As discussed below, the personalised plasma level metric may also include a ratio of the maximum plasma level, Cₘₐₓ, to the area under the plasma level time profile being less than a bleeding risk threshold.

In examples employing personalised target plasma level metrics, the patient data may include one or more target dependent patient parameters. The target dependent patient parameters may comprise one or more of: reported side effects (see “side effect monitoring” below); a patient thrombosis history; a patient haemorrhage history; a patient cancer history; a patient stroke history; a patient liver function metric; a patient heart function metric; a patient brain state; a patient smoking history; a patient alcohol history; a patient blood pressure; a patient mobility state; a patient menstruation state; a patient inflammation state; a patient infection state; a patient co-medication; a blood clotting metric; and a patient hydration state. The dosage calculation method may determine the personalised plasma level metric based on one or more of the target dependent patient parameters. The target dependent patient parameters may be monitored on an ongoing or regular basis and personalised target plasma level metrics may be refined on an ongoing basis as part of the ongoing treatment management discussed below.

In some examples, the method may determine personalised adjustments to the target plasma level metric based on specific calculations relating to one or more specific target dependent patient parameters, particularly those indicating a time-limited risk resulting from a risk event (fall, surgery etc). Other target dependent patient parameters that result in bleeding or haemorrhage risk may be chronic in nature or lifestyle dependent, such as a genetic clotting disorder, alcoholism etc.

In some examples, the method may output the personalised adjustments to the patient or HCP. For example, the method may indicate a personalised ideal therapeutic trough level for an individual.

### Risk Events

In some examples, the dosage calculation method may comprise determining the personalised plasma level metric based on a time since a bleeding risk event and/or a haemorrhage risk event. For example, the method may comprise applying a maximum adjustment to the nominal target plasma level metric immediately following the risk event and tapering the adjustment periodically back towards the nominal target plasma level metric as the time since the event increases. The method may indicate new dosage regimens with each variation of the adjustment.

For example, immediately following surgery, a patient may be at high risk of bleeding for the first 48 hours before the risk drops rapidly. Anticoagulants may be avoided during this period, except for patients at high risk of thrombosis. Following this initial period, the patient may be most at risk of thrombosis due to inflammation associated with healing and reduced mobility. At this stage, the method may determine a personalised target plasma level metric as a plasma trough level at the upper end of the ITL or above the ITL, e.g. 40 ng/ml. The dosage calculation method may comprise determining and indicating a dosage for administering to the patient that can provide the personalised target plasma level metric. Following a fixed period after the surgery, e.g. a number of days, the dosage calculation method may comprise reducing the personalised target trough level to 25-30 ng/ml and determining and outputting the dosage for administering accordingly. This may continue until the personalised target level metric is reduced to the ITL.

Other thrombosis risk events include: a thrombosis; one or more episodes of atrial fibrillation; dehydration; chemotherapy; an injury; a surgical procedure, particularly large joint orthopaedic procedures such as hip replacements; indwelling central venous catheters such as Hickman lines. Bleeding risk events include: a haemorrhagic stroke; an injury; a surgical procedure; needle penetration; or a patient fall.

A number of events result in both a bleeding risk and a thrombosis risk (e.g. falls and surgery). For such examples, the method may comprise determining the personalised plasma level metric as a ratio of Cₘₐₓ to the area under the plasma level time profile being less than a bleeding risk threshold. This may be in combination with maintaining the plasma trough level within the ITL. In such examples, the method may comprise determining and indicating a dosage regimen comprising a controlled release formulation or a dosage frequency of more than once daily, for example twice daily, combined with a lower dosage amount, for example 15 mg or less if suitable dosage forms are available. Although a higher dosage frequency can reduce compliance, it can be properly managed for carefully managed patients (e.g. cancer patients) and in care settings, such as post-surgery or in a nursing home. As mentioned above cancer patients can be at risk of both thrombosis and haemorrhage. Therefore, the method may also comprise determining similar personalised plasma level metrics (ratio and/or C_{trough} level) and dosage regimens for cancer patients. Furthermore, some patients may have a chronic bleeding or thrombosis risk and experience a thrombosis risk event or bleeding risk event. The method may also account for such dual risk by personalising the ratio and drug dosage regimen in a similar manner.

### Chronic or Lifestyle Related Risk Parameters

A patient fall may also provide an indication of a risk of further falls. There is a challenge in judging when the bleeding risk from a fall outweighs benefits of anticoagulation. A rough guide is if there are more than nine falls per year, anticoagulation may be contraindicated, but this depends on the nature of the fall. The dosage calculation method may reduce a target trough plasma level or the ratio of Cₘₐₓ to the area under the time profile based on the frequency and/or timing of falls as indicated by the patient fall history. Data from devices that measure fall propensity may input into the system and the patient facing app may offer tailored advice as to risks from falls, or other activities, mitigating actions and when, in relation to the timing of medication taking, their risks are highest and lowest.

The risks of bleeding are in part related to age. Reasons are not fully understood, but may in particular relate to the alterations in endothelium with age, including through changes in collagen. The dosage calculation method may reduce a target trough plasma level or the ratio of Cₘₐₓ to the area under the time profile based on patient age.

An additional problem for females relates to menstruation and contraception. Oestrogen increases the rate of thrombosis, so oestrogen-based pills are usually stopped. As an aside, the menstrual cycle can also affect INR readings making Warfarin a difficult drug for menstruating patients. A patient may then have heavy periods and become anaemic and then have blood transfusions. Some may inadvertently fall pregnant. Optimal management is missed for the majority. Most do not know if their periods are abnormal. Data on menstruation patterns may be inputted to the system and the patient facing app may offer tailored advice as to contraception and menstruation, e.g. advise appropriate treatment for heavy menstrual bleeding. The app could also alert the HCP to the issue.

Other target dependent patient parameters that can increase bleeding risk and therefore may require a reduction in target trough plasma level and/or the ratio of Cₘₐₓ to the area under the time profile include: high patient blood pressure (haemorrhagic stroke risk), poor liver function, the presence of amyloid in the brain, poor renal function, high alcohol consumption and an indication of aspirin, clopidogrel, NSAIDs, P-gp inhibitors, steroids, antidepressants or any other medication that can result in heightened bleeding risk. In some examples, the dosage calculation method may determine a bleeding risk score based on the presence, timing, and/or severity of a bleeding risk event and one or more of the aforementioned bleeding risk factors. The dosage calculation method may use the HAS-BLED score to determine the bleeding risk score.

Alcohol and caffeine intake can also increase AF and dysthymias, potentially increasing the chance of strokes and thrombosis.

The dosage calculation method may determine a personalised target plasma level metric by decreasing the target trough plasma level if the bleeding risk score exceeds a bleeding risk threshold.

Other target dependent patient parameters that can increase thrombosis risk and therefore may require an increase in target trough plasma level include: genetic profile;

active cancer or inflammatory state; and patient hydration. In some examples, the dosage calculation method may determine a thrombosis risk score based on the presence, timing, and/or severity of a thrombosis risk event and one or more of the aforementioned thrombosis risk parameters.

The dosage calculation method may determine a personalised target plasma level metric by increasing the target trough plasma level if the thrombosis risk score exceeds a thrombosis risk threshold.

In some examples, the dosage calculation method may receive the bleeding risk score and/or the thrombosis risk score via data entry from an HCP.

The dosage calculation method may determine a personalised target plasma level metric as a ratio of Cₘₐₓ to the area under the plasma level time profile being less than a bleeding risk threshold if the thrombosis risk event exceeds a thrombosis risk threshold and the bleeding risk score exceeds a bleeding risk threshold.

In some examples, the method may comprise assigning patients to one of a plurality of sub-groups. Each sub-group may correspond to one or more of the target dependent patient parameters described above. The method may comprise assigning the patient based on time since a risk event, and/or the presence of a particular patient risk factor. Each sub-group may have a corresponding personalised target plasma level metric.

### Ongoing Treatment Management

As the patient's state changes, so does their pharmacokinetics and pharmacodynamics, with the result that they can become underdosed or overdosed.

Ideally, the patient should be reviewed at least once per year and for high-risk patients potentially every 3 to 6 months, or more frequent follow-ups for particularly high-risk patients (e.g. those with renal failure, where signs of bleeding are occurring in preparation for surgery, when the subject is taking drugs with interactions on absorption or where the chance of stroke is particularly high).

A comprehensive DOAC monitoring approach may include one or more of the following items assessed at each follow-up:
1. Current health status
   e.g. a measure of blood pressure and blood sample to check for new anaemia, liver and kidney function.
2. Lifestyle assessment, including the nature of sports.
3. Risks of falls.
4. Personalised risk of haemorrhage and thrombosis.
5. Adherence and cost.
6. Bleeding/thromboembolic events.
7. Adverse effects.
8. Complete medication review for drug interactions.
9. Reassessment of appropriateness of therapy.
10. Repeat warranted laboratory parameters.
11. Continuing follow-up appointments for patients.

Embodiments of the digital app and associated methods may include providing a HCP with a summary of key patient data parameters that have been tracked during the treatment period. The digital app may track any of the patient data parameters described herein for presentation to an HCP. The patient data parameters may be tracked via manual data entry from a patient, HCP, or clinic, from clinical records, or any other input method. Parameters which may be tracked include, amongst others, age, weight, renal function, liver function, bleeding tendencies, any new thrombotic events and falls risks.

Embodiments of the digital app and associated methods may include tracking the aforementioned patient data parameters as updated patient data (for example on a regular basis (daily, weekly, monthly)), processing the updated patient data parameters to determine an updated dosage for administering to the patient; and indicating the updated dosage. For example, changes to the patient data underpinning the plasma level prediction model (such as kidney function or medication) may result in a different calculated dosage to obtain the target plasma level metric. As a further example, changes to the patient data that indicate an increase in bleeding and/or thrombosis risk may result in a personalisation of the target plasma level metric (as described in the preceding section), thereby resulting in a change to the recommended dosage for administering to the patient. Indicating the updated dosage may comprise: indicating the updated dosage to a HCP at a regular review meeting; and/or indicating the updated dosage to a HCP or other healthcare professional (pharmacist, nurse etc) via an alert if a change in the updated dosage exceeds an alert threshold.

Embodiments of the digital app and associated methods may include tracking or monitoring patient data comprising side effects such as excessive bleeding, GI dysfunction, vomiting, skin rashes etc, as well as the behavioural pattern of the patient (and others listed in side effect monitoring below). The patient data may be provided to the HCP as part of the health care review or alerted if significant, e.g. haemorrhage.

By tracking the patient data, underlying changes to a patient's physiology with time, age, disease progression or through drug or lifestyle or other environmental factors can be periodically evaluated to maintain personal calibration of the dose yielding ideal therapeutic level for that patient.

### Blood Coagulation Test Monitoring

Ideally an ongoing monitoring program should include measuring drug trough plasma levels. However the measurement of plasma drug concentrations in both hospitals and the Home Care Provider setting is costly and seldom undertaken.

Specific quantitative measures exist for edoxaban such as plasma edoxaban drug concentration, as well as coagulation measurements such as anti-Xa activity and prothrombin time (PT), which can be used to calculate the approximate plasma level, for example, to indicate when the bleeding risk is sufficiently low to allow surgery, with a typical threshold being a blood level of ˜30 ng/ml. One issue is that these measures have not been shown to directly relate to clinical outcome and agreed standardized therapeutic ranges have not been established. In addition, these more specific tests are not always available to every healthcare provider. Furthermore, the reference ranges associated with each coagulation measurement may depend on the specific assay kit used and the indication. However, unlike with the vitamin K antagonist anticoagulants, these tests can relate directly in time to the circulating edoxaban plasma levels due to the lack of PK-PD hysteresis.

From the CDER Clinical Pharmacology and Biopharmaceutics Review for edoxaban tosylate, relationships between drug plasma levels Cp and coagulation measures can be calculated, and the following equations derived:

Anti−Xa activity(IU/ml)=0.0136·Cp Equation 14:

Anti−Xa activity(IU/ml)=0.0305·Cp−3.398 Equation 15:

Prothrombin time (sec)=0.0483·Cp+15 Equation 16:
where Cp is the plasma edoxaban concentration (ng/ml). Equation 14 is used when Cp is in the range 0-200 ng/ml, while Equation 15 is used when Cp is greater than 200 ng/ml.

In addition, new assays are in development that may enable better computation of edoxaban drug plasma levels.

Embodiments of the digital app or associated methods may include receiving patient data comprising a patient coagulation metric (also referred to herein as a patient clotting metric) from a blood coagulation test result. The patient clotting metric may comprise a drug plasma level (also referred to as drug concentration). The patient clotting metric may comprise any of the above blood clotting metrics and the embodiments may comprise calculating a measured drug plasma level based on the blood clotting metric. In some examples, the embodiments include calibrating the dosage calculator and/or plasma level prediction model by comparing the measured drug plasma level to a corresponding plasma level metric calculated by the dosage calculator. Following calibration, embodiments may comprise processing the patient data with the calibrated dosage calculator to determine an updated dosage of edoxaban for administering to the patient. Personalised calibration can advantageously help account for the inter-subject variability illustrated in FIG. 4G

A challenge with measuring drug or clotting level via a blood test is relating this to a time of administration and therefore a trough level is typically taken. This can be logistically challenging compared to taking a sample at any time point. Embodiments of the present disclosure allow for individualised calculation of clotting parameters and drug profile by recording the time of medication and the time of blood sample collection (which may be any time) as patient data. Embodiments of the digital app and associated methods may include: receiving a time of drug administration, a time of blood sample and a measured drug plasma level as patient data; and combining the measured drug plasma level, the time of drug administration, and the time of the blood sample with the plasma level time profile estimated by the dosage calculator to adapt the measured drug plasma level to a measurement-derived maximum plasma level, Cₘₐₓ, a measurement-derived average plasma level, C_{average}, or a measurement derived trough plasma level, C_{trough}. In other words, the digital app and associated methods can convert a blood test taken at any time point to a measurement-derived maximum plasma level or a measurement-derived trough plasma level.

Embodiments of the digital app and associated methods may also calibrate the plasma level prediction model and/or dosage calculator based on the measured drug plasma level or clotting metric. Embodiments of the digital app and associated methods may include: receiving a time of drug administration and a time of blood sample as patient data; estimating a drug plasma concentration at the time of the blood sample using the dosage calculator based on the dosage amount and the time of drug administration; and calibrating the plasma level prediction model and/or the dosage calculator based on the difference between the estimated drug plasma concentration and the measured drug plasma concentration. In some examples, embodiments may comprise calibrating the plasma level prediction model and or dosage calculator based on a difference between the maximum plasma level estimated by the dosage calculator and the measurement-derived maximum plasma level and/or a difference between the trough plasma level estimated by the dosage calculator and the measurement-derived trough plasma level. In this way, the dosage can be adjusted to obtain a corrected ITL. Using both trough and maximum levels of coagulation, the best dose for a particular individual can be recommended.

In examples where the digital app and associated methods are used for patients having a high bleeding and clotting risks (e.g. surgery patients, cancer patients, elderly patients, patients prone to falls), regular blood test monitoring may be employed to ensure accuracy of the dosage calculator and/or underlying plasma level prediction model. The methods may comprise updating the dosage calculator, the plasma level prediction model, the personalised target plasma level metrics (e.g. ratio or trough level as described in previous section), and/or the indicated dosage for administering to the patient, based on the calculated or measured drug plasma level.

The availability of coagulation test data also allows for personal calibration of the dose prediction model (and can help account for the inter-subject variability illustrated in FIG. 4G). The plasma level prediction model and dosage calculator can predict circulating drug for an average patient at any time following drug intake. This would lead to a predicted coagulation test result. By comparing the actual coagulation test result to the predicted, the dose prediction model can be proportionately adjusted for the individual characteristics or vice versa.

If the dose response is personally calibrated with confidence, then dosing may be adjusted to accommodate acute or long-term changes in patient data, such as adjustment to accommodate temporary or long-term antiplatelet therapy, for example given following coronary artery stenting (e.g. via personalised target plasma level metrics as described above). Drugs like aspirin or NSAIDs may also be taken inadvertently by a patient for a headache or pain, the patient not realising it has an additive effect with edoxaban on the risk of a major bleed.

It will be appreciated that personal calibration via blood tests is optional. In many examples (e.g. lower risk patients), the HCP may rely on the dosage recommendation and/or plasma level metrics provided by the app and associated methods disclosed herein.

Other patient data parameters that may be monitored by the app or associated methods may include signs and symptoms of bleeding, complete blood count, and a comprehensive metabolic panel specifically evaluating liver function tests, albumin, total bilirubin, and serum creatinine.

### Side Effect Monitoring

As noted above, the app and associated methods may comprise receiving patient data as self-reported side effects. The digital app and associated methods may monitor side effects of edoxaban, including those that can occur when the dosage is too high (see Table 4), using suitable questions. GI tract disturbances and bleeding in particular may be closely monitored. The digital app and associated methods may take a number of actions in response to detecting a side effect above a respective sensitivity level, including: alerting a clinician; advising the patient to make a medical appointment; recommending a blood test for calibration (see “Ongoing Treatment Management” section above); adjusting a bleeding risk level; adjusting a thrombosis risk level; adjusting a personalised target plasma level metric; recalculating the dosage for administering to the patient based on the adjusted bleeding risk level, adjusted thrombosis risk level and/or adjusted personalised target plasma level metric; and indicating an updated dosage for administering to the patient to the clinician or patient. By monitoring and acting on side effects the app and methods can provide important feedback to the HCP on the need to obtain measures of anticoagulation (blood tests) and advise on suitable dosage change for that particular patient.

TABLE 4
Common Side Effects of edoxaban

Abdominal pain
Anaemia
Dizziness
Haemorrhage
Headache
Nausea
Skin reactions
Abnormal liver function tests
CNS haemorrhage (uncommon)
Thrombocytopenia (uncommon)

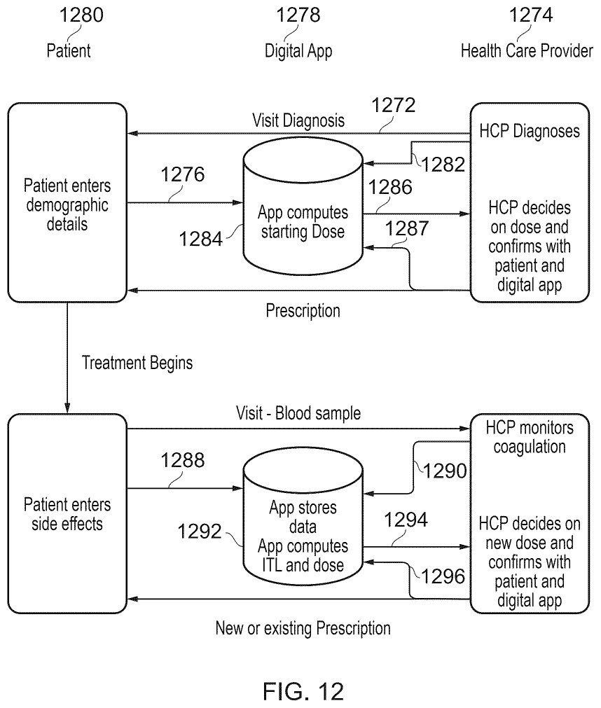

FIG. 12 illustrates an example patient monitoring process according to an embodiment of the present disclosure.

Following a diagnosis 1272 by a health care provider (HCP) 1274, for example a HCP, a first step of the method comprises receiving 1276, at the digital app 1278, patient data comprising demographic details entered by the patient 1280. The method may also comprise receiving 1282, at the digital app, patient data from the HCP 1274 (e.g. via manual data entry or clinical records etc).

Following receipt of the patient data, the method proceeds to processing the patient data 1284 using the dosage calculator to determine a starting dosage regimen for administering to the patient 1280. Following calculation of the starting dosage regimen, the method comprises indicating the dosage regimen 1286 to the HCP 1274. The HCP 1274 may consider the starting dosage regimen calculated by the app 1278 and provide an initial prescription to the patient 1280. The prescription may be provided 1287 to the digital app 1278 as patient data.

The patient 1280 begins treatment. During treatment the method comprises receiving updated patient data 1288 from the patient 1280, such as side effects or events, and receiving updated patient data 1290 from the HCP following physiological tests such as blood test results comprising blood clotting metrics, drug plasma levels and/or CrCL. The method proceeds to process the updated patient data 1292 to determine an updated dosage for administering to the patient. Processing the updated patient data 1292 may comprise calibrating the plasma level prediction model and/or the dosage calculator based on the measured clotting metric or drug plasma level as described above. Processing the updated patient data 1292 may comprise calculating an updated dosage regimen based on a significant change in a patient data parameter driving the calculator, such as a change in CrCL. Processing the updated patient data 1292 may also comprise personalising the target plasma level metric and calculating an updated dosage regimen as described above. The method proceeds to indicating 1294 the updated dosage regimen to the HCP 1274.

The HCP may consider the updated dosage regimen calculated by the app 1278 and provide an updated prescription to the patient 1280. The updated prescription may be provided 1296 to the digital app 1278 as further updated patient data. The lower loop illustrated on the Figure may be repeated as treatment progresses and optionally as the patient makes further or regular visits to the HCP 1274 e.g. for further blood tests.

### Patient Instruction and Education

The digital app and associated methods may comprise providing instruction, guidance and education to the patient to support them with their ongoing edoxaban medication. Due to a lack of ongoing review at present, many patients can feel uncertain in relation to their medication which can lead to poor compliance. For example, the consequences of a thrombosis and a DOAC prescription for the patient extends well beyond the direct effects. Patients live with lots of fear and do not know what to do if a further bleed happens. There are two phases to management of venous thromboembolism, for example the active phase and the preventative phase. In the first phase, the aim is to stop clot extension and early recurrence. This lasts one month, perhaps extending in some to 3 months. After this, there is more flexibility in treatment. There is also limited patient understanding of how to take their medication, with many not realising that they need to request a repeat prescription, interpreting their hospital script as all that is required, like for a course of antibiotics. Some may overlook a requirement for certain medications to be taken with food.

Many patients do not know what to do if they develop a further clot and wrongly assume that they should carry out preventative actions, for example with a clot in their leg, moving the leg around, whereas they should do the opposite, keeping the leg elevated and immobile. Many patients have local complications from a thrombosed vein, with a postphlebitic limb very common. This is often badly managed. The nature and timing of how to wear compression stockings and bandages is not known by most. For patients who have had pulmonary emboli, many misinterpret subsequent chest pain and shortness of breath. If benign causes are misinterpreted, this leads to anxiety and exercise avoidance, with consequent deconditioning and increased future risk of cardiopulmonary events.

Similar concerns and uncertainties around a edoxaban prescription may be experienced by patients taking the drug for prophylaxis purposes, such as patients with AF.

The fact that DOACs aren't monitored in the way that warfarin is with a warfarin clinic means there is limited opportunity for education. Overall, it is estimated that 20 to 25% of patients are not taking their originally prescribed dose.

Therefore, as outlined below, the methods and apparatus of the disclosure are intended to provide patient instruction, guidance and education to support patients with their medication thereby improving trust and compliance.

### Instructing Dose Administration

Embodiments of the digital app and associated methods may comprise indicating the dosage to the patient via a patient device. The app may indicate the dosage amount to the patient and may at the same time provide information on how and when to take the medication in relation to food, how to address missed dosing and/or if there are issues with taking other drugs. Taking DOACs with food can slow the absorption of the drug by as much as three hours and the app may instruct the patient accordingly. Compliance is an issue with edoxaban and anticoagulants since failure to take a dose can lead to a reduction in the activity. The digital app can reinforce the need to take the medication on a regular basis according to HCP recommendations, through the use of visual, audio and/or tactile prompts, alerts, alarms, reminders and other alert mechanisms from the patient device. For missed doses, the app and associated methods may calculate a new schedule of dosing and/or adjusted dosage amounts (up or down) for a temporary period using the dosage calculator and the target plasma level metric. The app and associated methods may comprise: receiving a new candidate comedication (from a HCP or patient), comparing the comedication against a reference comedication list; and indicating a comedication instruction. The comedication instruction may comprise: a revised dosage for edoxaban, an instruction not to take the comedication; and/or an instruction to consult the HCP.

### Side Effect Guidance

As noted above, the app and associated methods may comprise receiving patient data as self-reported side effects. The digital app and associated methods may provide guidance to the patient on the reported side-effects or any other side effects to allay patient concern. As noted above, the app may alert the patient that they should seek medical care in the case of severe side effects.

### Lifestyle and Risk Guidance

The digital app and associated methods may also incorporate and deliver a comprehensive education program with advice on the self-monitoring of side effects (as described above), messaging relating to precautions to avoid the chance of stroke and lifestyle modification improvements, such as smoking cessation, reduction in alcohol consumption, journey durations and habits, and hydration status. As noted above in the outputs section, the app and methods may determine periods of bleeding risk and/or thrombosis risk based on the plasma level time profile and suggest timing of risk activities (sport, long journeys etc) relative to the medication time and maximum plasma level, Cₘₐₓ, as indicated by the plasma level time profile. In this way, the app and methods can minimise the risk of thrombosis and haemorrhage while enabling the patient to maintain a quality of life. As also noted above, the app and methods may calculate and indicate an adjusted dose in advance of and following high risk procedures (e.g. surgery and long journeys).

The education advice may include instructions on management of a phlebitic limb, including duration of elevation, type of bandage or stocking and application procedure, with tracking of any unwanted consequences and management thereof.

The app and methods may also advise on risks of various activities, in particular contact sports and those with higher risks of falls such as skiing. The app and methods may provide individualised risks for informed patient decisions. For example, the app may advise on strategies to decrease risk during long journeys.

The app and methods may provide advice on patients regarding decrease in haemorrhage risk from lifestyle changes, including improving balance to prevent falls and type of exercise, whilst still ensuring optimal quality of life which could be impacted if activities are curtailed too much.

The app and methods may track respiratory symptoms in patients who have had a pulmonary embolism to help identify whether chest pain and shortness of breath is merely residual effects of the original embolus or is a new cause for concern. The prospective tracking overcomes the problems of patient memory.

For females, the app and methods may advise on contraception and also management of heavier periods from the anticoagulant. The app and methods may incorporate bleeding scores to help determine whether periods are abnormal and warrant additional investigation, to pick up, for example, a new cancer.

For patients who have falls, the app and methods may track the number of falls and their type to help with risk management.

The example, dosage calculators, plasma level prediction models and methods for calculating personalised target metrics described herein are exemplary. Treatment algorithms, including computation of ideal and personalised plasma level metrics, may also be expressed by explicit rules (e.g. if . . . then . . . ), Bayesian or other statistical inference derived from population data, or via Machine Learning (e.g. Deep Learning). The type of algorithm may be selected for performance, suitability for context and governing regulatory framework. In some examples, dosage calculators and/or plasma level prediction models may be refined continuously as new data is accumulated or revised periodically for regulatory approval according to governance requirements and patient risk.

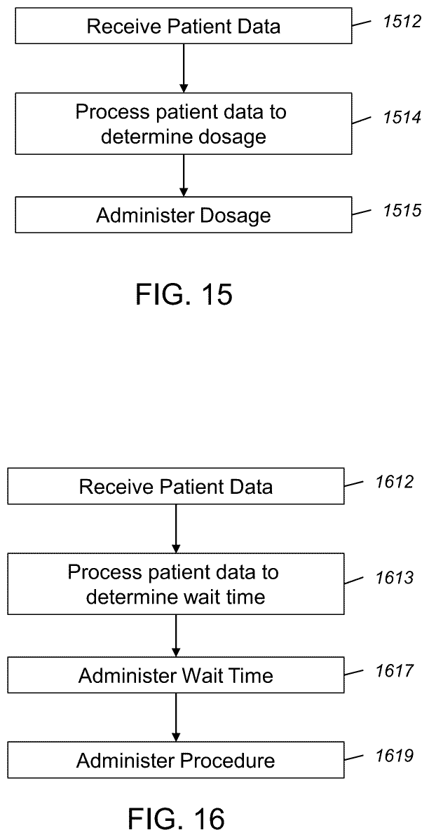

FIG. 15 illustrates a method for administering a dosage of edoxaban to a patient for the treatment or prevention of thrombosis.

A third step 1515 of the method comprises administering a dosage of edoxaban to the patient. The dosage is determined by: a first step 1512 comprising receiving patient data relating to a patient, wherein the patient data includes a kidney function metric of the patient; and a second step 1514 comprising processing, using one or more processors, the patient data with a dosage calculator to determine the dosage of edoxaban for the patient, wherein the dosage calculator is derived from a plasma level prediction model that predicts edoxaban drug plasma levels, and the dosage calculator determines the dosage for the patient based in part on the kidney function metric of the patient.

FIG. 16 illustrates a method for determining and administering a procedure wait time for a patient following withdrawal of a direct oral anti-coagulant (DOAC) in advance of an invasive procedure.

A first step 1612 comprises receiving patient data relating to a patient, wherein the patient data includes a kidney function metric of the patient and a DOAC dosage for the patient.

A second step 1613 comprises processing, using one or more processors, the patient data with a dosage calculator to determine the procedure wait time for a drug plasma level to fall below an invasive procedure plasma level threshold, wherein the dosage calculator is derived from a plasma level prediction model that predicts DOAC drug plasma levels, and the dosage calculator determines the procedure wait time for the patient based in part on the kidney function metric of the patient

A third step 1617 comprises administering the procedure wait time in advance of the invasive procedure to reduce a risk of haemorrhage during the invasive procedure.

An optional fourth step 1619 comprises administering or performing the invasive procedure on the patient.

Throughout the present specification, it will be appreciated that any reference to “close to”, “before”, “shortly before”, “after” “shortly after”, “higher than”, or “lower than”, etc, can refer to the parameter in question being less than or greater than a threshold value, or between two threshold values, depending upon the context.
[1]: PARASRAMPURIA, D. A. & TRUITT, K. E. 2016. Pharmacokinetics and Pharmacodynamics of Edoxaban, a Non-Vitamin K Antagonist Oral Anticoagulant that Inhibits Clotting Factor Xa. Clin Pharmacokinet, 55, 641-55.
[2]: CENTER FOR DRUG EVALUATION AND RESEARCH 2014. Edoxaban Tosylate Clinical Pharmacology and Biopharmaceutics Review.
[3]: US FOOD AND DRUG ADMINISTRATION 2015. SAVAYSA Prescribing Information.
[4]: SALAZAR, D. E., MENDELL, J., KASTRISSIOS, H., GREEN, M., CARROTHERS, T. J., SONG, S., PATEL, I., BOCANEGRA, T. S., ANTMAN, E. M., GIUGLIANO, R. P., KUNITADA, S., DORNSEIF, B., SHI, M., TACHIBANA, M., ZHOU, S. & ROHATAGI, S. 2012. Modelling and simulation of edoxaban exposure and response relationships in patients with atrial fibrillation. Thromb Haemost, 107, 925-36.
[5]: SONG, S., KANG, D., HALIM, A. B. & MILLER, R. 2014. Population pharmacokinetic-pharmacodynamic modeling analysis of intrinsic FXa and bleeding from edoxaban treatment. J Clin Pharmacol, 54, 910-6.
[6]: YIN, O. Q., TETSUYA, K. & MILLER, R. 2014. Edoxaban population pharmacokinetics and exposure-response analysis in patients with non-valvular atrial fibrillation. Eur J Clin Pharmacol, 70, 1339-51.
[7]: NIEBECKER, R., JÖNSSON, S., KARLSSON, M. O., MILLER, R., NYBERG, J., KREKELS, E. H. J. & SIMONSSON, U. S. H. 2015. Population pharmacokinetics of edoxaban in patients with symptomatic deep-vein thrombosis and/or pulmonary embolism—the Hokusai-VTE phase 3 study. British Journal of Clinical Pharmacology, 80, 1374-1387.
[8]: KREKELS, E. H., NIEBECKER, R., KARLSSON, M. O., MILLER, R., SHIMIZU, T., KARLSSON, K. E., RUFF, C. T., SIMONSSON, U. S. & JONSSON, S. 2016. Population Pharmacokinetics of Edoxaban in patients with non-valvular atrial fibrillation in the ENGAGE AF-TIMI 48 Study, a Phase III Clinical Trial. Clin Pharmacokinet, 55, 1079-90.
[9]: WEITZ, J. I., CONNOLLY, S. J., PATEL, I., SALAZAR, D., ROHATAGI, S., MENDELL, J., KASTRISSIOS, H., JIN, J. & KUNITADA, S. 2010. Randomised, parallel-group, multicentre, multinational phase 2 study comparing edoxaban, an oral factor Xa inhibitor, with warfarin for stroke prevention in patients with atrial fibrillation. Thromb Haemost, 104, 633-41.

1. A method for administering a dosage of edoxaban to a patient for the treatment or prevention of thrombosis, the method comprising:
administering a dosage of edoxaban to the patient, wherein the dosage of edoxaban is determined by:
receiving patient data relating to a patient, wherein the patient data includes a kidney function metric of the patient; and
processing, using one or more processors, the patient data with a dosage calculator to determine the dosage of edoxaban for administering to the patient, wherein the dosage calculator is derived from a plasma level prediction model that predicts edoxaban drug plasma levels, and the dosage calculator determines the dosage for the patient based in part on the kidney function metric of the patient.

2. The method of claim 1, wherein the patient data further comprises one or more of: a patient age; a patient ethnicity; a patient sex; a patient weight; a patient genotype; a patient cardiac metric; and a patient medication list.

3. The method of claim 2, wherein, the patient medication list comprises an indication of whether the patient is consuming one or medications comprising: a proton-pump inhibitor; a calcium channel blocker; a P-gp inhibitor; an antifungal; an antiarrhythmic drug; an antibiotic; or a drug that increases a bleeding risk including a drug with an anticoagulant effect.

4. The method of claim 1, wherein the patient data further comprises one or more of: reported side effects; alcohol intake; smoking history, a patient clotting metric; a treatment purpose; patient genetic determinants; patient co-conditions; a patient activity level; a patient dosage compliance; a patient liver function; a patient thrombosis history; a patient haemorrhage history; a patient cancer history; a family thrombosis history; familial stroke history, familial bleeding history; cardiovascular history; metabolic history; a patient blood pressure history; a patient platelet count; a patient heart rate; and a patient haematocrit.

5. The method of claim 1 further comprising:
receiving updated patient data; and
processing the updated patient data with the dosage calculator to determine an updated dosage; and
indicating the updated dosage.

6. The method of claim 5, wherein the updated patient data includes a patient clotting metric and/or a drug concentration, from a blood test result and wherein the method further comprises calibrating the dosage calculator by adjusting the dosage calculator and/or the plasma level prediction model using the patient clotting metric and/or drug concentration.

7. The method of claim 1, wherein processing the patient data with a dosage calculator to determine the dosage of edoxaban for administering to the patient comprises:
receiving a target plasma level metric; and
calculating the dosage for administering to the patient by processing the patient data and the target plasma level metric with the dosage calculator.

8. The method of claim 1, wherein processing the patient data with a dosage calculator to determine the dosage of edoxaban for administering to the patient comprises:
setting an initial value of a dose estimate;
processing the dose estimate with the dosage calculator to estimate a plasma level metric;
comparing the plasma level metric to a target plasma level metric; and
determining the dosage for administering to the patient by refining the dose estimate based on the comparison.

9. The method of claim 7, wherein the target plasma level metric comprises one or more of:
a target trough plasma level comprising an ideal therapeutic level;
a target maximum plasma level being less than a maximum level threshold;
a target average plasma level over a dosing interval at steady state comprising an ideal therapeutic level;
a target area under the curve of a plasma level time profile comprising an ideal therapeutic level;
a target ratio of a maximum plasma level to a trough plasma level comprising an ideal therapeutic ratio and level; or
a target ratio of the maximum plasma level to the area under the curve of the plasma level time profile comprising an ideal therapeutic ratio and level.

10. The method of claim 7, wherein the patient data comprises one or more target dependent patient parameters and wherein the method comprises:
determining the target plasma level metric as a personalised target plasma level metric based on the one or more target dependent patient parameters.

11. The method of claim 10 comprising:
determining a bleeding risk score for the patient based on one or more of the target dependent patient parameters;
determining a thrombosis risk score for the patient based on one or more of the target dependent patient parameters; and
determining the personalised target plasma level metric based on the bleeding risk score and/or the thrombosis risk score.

12. The method of claim 10, wherein the personalised target plasma level metric comprises:
a trough plasma level or an average plasma level, comprising a personalised adjustment to an ideal therapeutic level based on the one or more target dependent patient parameters; and/or
a ratio of a maximum plasma level to an area under a plasma level time profile being less than a bleeding risk threshold.

13. The method of claim 1, wherein the dosage calculator comprises a machine learning algorithm trained using the plasma level prediction model.

14. The method of claim 13, wherein the machine learning algorithm comprises an adjustable machine learning algorithm and the method further comprises:
receiving updated patient data including a patient clotting metric from a blood test result; and
adjusting the machine learning model based on the patient clotting metric.

15. The method of claim 1, wherein processing the patient data with the dosage calculator to determine the dosage of edoxaban for administering to the patient comprises:
processing the patient data with the dosage calculator to determine an ideal dosage regime; and
selecting the dosage for administering to the patient from a selection of available dosage regimes based on the ideal dosage regime.

16. The method of claim 15, wherein the selection of available dosage regimes comprise dosage amounts comprising: 15 mg, 30 mg, or 60 mg of edoxaban.

17. The method of claim 15, wherein the selection of available dosage regimes comprise dosage amounts comprising:
any multiple of 0.5 mg;
any multiple of 1 mg;
any multiple of 5 mg;
any multiple of 10 mg; or
any multiple of 25 mg.

18. The method of claim 1, wherein processing the patient data with the dosage calculator to determine the dosage of edoxaban for administering to the patient comprises processing the patient data with the dosage calculator to determine one or more of: a dosage amount; a dosage time; a dosage frequency; and/or a dosage type, wherein the dosage type comprises an edoxaban slow-release formulation with a specific release time.

19. A method for determining and administering a procedure wait time for a patient following withdrawal of edoxaban in advance of an invasive procedure, the method comprising:
receiving patient data relating to a patient, wherein the patient data includes a kidney function metric of the patient and an edoxaban dosage for the patient;
processing, using one or more processors, the patient data with a dosage calculator to determine the procedure wait time for a drug plasma level to fall below an invasive procedure plasma level threshold, wherein the dosage calculator is derived from a plasma level prediction model that predicts edoxaban drug plasma levels, and the dosage calculator determines the procedure wait time for the patient based in part on the kidney function metric of the patient; and
administering the procedure wait time in advance of the invasive procedure to reduce a risk of haemorrhage during the invasive procedure.

20. The method of claim 19, wherein after administering the procedure wait time, the method comprises administering the invasive procedure on the patient.
